(19) United States
(12) Patent Application Publication
(10) Pub. No.: US 2006/0166989 A1
(43) Pub. Date: Jul. 27, 2006

(54) 2-naphthamide derivatives

(75) Inventors: Makoto Shimazaki, Kyoto-fu (JP); Osamu Sakurai, Kyoto-fu (JP); Toshiki Murata, Nara-ken (JP); Klaus Urbahns, Lund (SE); Noriyuki Yamamoto, Osaka-fu (JP); Satoru Yoshikawa, Kanagawa-ken (JP); Masaomi Umeda, Shiga-ken (JP); Masaomi Tajimi, Aichi-ken (JP); Tsutomu Masuda, Aichi-ken (JP); Takuya Shintani, Kyoto-fu (JP); Haruka Shimizu, Tokyo (JP)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Healthcare AG, Leverkusen (DE)

(21) Appl. No.: 10/517,677

(22) PCT Filed: May 30, 2003
(86) PCT No.: PCT/EP03/05705

§ 371 Date: Jul. 11, 2005

(30) Foreign Application Priority Data
Jun. 12, 2002 (GB) 0213488.0

(51) Int. Cl. A61K 31/537; A61K 31/44; A61K 31/195; C07D 213/55; C07D 265/30; C07C 237/40

(52) U.S. Cl. 514/237.5; 514/357; 514/563; 544/162; 546/335; 562/450

(57) ABSTRACT

The present invention relates to 2-naphthamides, which are useful as an active ingredient of pharmaceutical preparations. The 2-naphthamides of the present invention have IP receptor antagonistic activity, and can be used for the prophylaxis and treatment of diseases associated with IP receptor activity. Such diseases include urological diseases or disorder as follows: bladder outlet obstruction, overactive bladder, urinary incontinence, detrusor hyper-reflexia, detrusor instability, reduced bladder capacity, frequency of micturition, urge incontinence, stress incontinence, bladder hyperreactivity, benign prostatic hypertrophy (BPH), pro-statitis, urinary frequency, nocturia, urinary urgency, pelvic hypersensitivity, urethritis, pelvic pain syndrome, prostatodynia, cystitis, or idiophatic bladder hypersensitivity. The compounds of the present invention are also useful for treatment of pain including, but not limited to inflammatory pain, neuropathic pain, acute pain, chronic pain, dental pain, premenstrual pain, visceral pain, headaches, and the like; hypotension; hemophilia and hemorrhage; and inflammation, since the disease is also alleviated by treatment with an IP receptor antagonist.

### TECHNICAL FIELD

The present invention relates to a 2-naphthamides which are useful as an active ingredient of pharmaceutical preparations. The 2-naphthamides of the present invention have an IP receptor antagonistic activity, and can be used for the prophylaxis and treatment of diseases associated with IP receptor activity.

More specifically the 2-naphthamide derivatives of the present invention are useful for treatment and prophylaxis of urological diseases or disorders.

The compounds of the present invention are also useful for treatment of pain; hypotension; hemophilia and hemorrhage; inflammation; respiratory states from allergies or asthma, since the disease also is alleviated by treatment with an IP receptor antagonist.

### BACKGROUND ART

Prostaglandins (or prostanoids, PGs) are a group of bioactive lipid mediators generated from membrane phospholipids. They are formed from 20-carbon essential fatty acids containing 3, 4, or 5 double bonds, and carry a cyclopentane ring. They are divided into 6 main classes D, E, F, G, H or I) by the cyclopentane ring structure. The main classes are farther subdivided by subscripts 1, 2, or 3, reflecting their fatty acid precursors. PGI2 is a member of prostanoids, and it has a double ring structure and is derived from arachidonic acid. The receptor for PGI2 is a seven transmembrane G-protein coupled receptor, called IP receptor. IP receptor couples at least to Gs-type G-protein, and activates adenylate cyclase and phospholipase C. The expression of IP receptor is demonstrated in aorta, coronary/pulmonary/cerebral arteries, platelets, lung, and dorsal root ganglions in addition to several other tissues.

One of the well-known actions of PGI2 on blood vessels is to cause vasodilation and hypotension. Especially in septic shock, PGI2 is produced and participate in the induction of systemic hypotension (G. D. Bottoms et al, Am J Vet Res 1982, 43(6), 999-1002). Therefore, IP receptor antagonists may prevent hypotension associated with septic shock.

Another well-known action of PGI2 on platelets is to suppress aggregation. In the IP receptor knock out mice, FeCl₃-induced thrombosis formation was enhanced in comparison with that in wild type mice (T. Murata et al, Nature 1997, 388, 678-682.), confirming the involvement of IP receptor in the platelet inhibition. Therefore, IP receptor antagonists may enhance the platelet activation and suppress excessive bleeding such as, but not limited to, hemophilia and hemorrhage.

PGI2 also participate in the inflammation. In the inflamed tissue, various inflammatory mediators, including prostaglandins, are produced. PGI2 is also generated and induces vasodilation to increase blood flow. This enhances vascular permeability, edema formation and leukocyte inflammation in the inflamed region (T. Murata et al, Nature 1997, 388, 678-682.). Therefore, IP receptor antagonists may be efficacious for the treatment of inflammation.

PGI2 may be involved in the pathogenesis of respiratory allergy or asthma. It is spontaneously generated and the major prostaglandin in human lung, and the appropriate antigen challenge increases PGI2 production (E. S. Schulman et al, J Appl Physiol 1982, 53(3), 589-595.). Therefore, IP receptor antagonists may have a utility for the treatment of those respiratory diseases.

In addition, an important role of IP receptor in the induction of hyperalgesia has been clearly shown by IP receptor knockout mice (T. Murata et al., Nature 1997, 388, 678-682.). Injection of acetic acid into the peritoneal cavity induced production of PGI2. This PGI2 is considered to bind to IP receptor on sensory neurons. As IP receptor couples to the activation of both adenylate cyclase and phospholipase C, cAMP-dependent protein kinase (PKA) and protein kinase C (PKC) are activated. PKA and PKC are known to modulate ion channels on sensory neurons such as VR1, P2X3, and TTX-P As a result, PGI2 sensitizes sensory neurons to enhance the release of neurotransmitters. Hence, acetic acid injection induces nociceptive response (writhing) in mice. This acetic acid-induced writhing was greatly reduced in PGI2 receptor-null mice as the same level as indomethacin-treated wild type mice. Several other in vivo hyperalgesia studies in rodents and in vitro studies further support that PGI2 plays a major role in the induction of hyperalgesia and that PGI2 acts as important modulator of sensory neurons (K. Bley et al, Trends in Pharmacological Sciences 1998, 19(4), 141-147.). Therefore, IP receptor antagonists may be useful for the treatment of pain.

Sensory neurons play very important roles not only in the pain sensation but also in the sensation of bladder distension. In normal subjects, A-delta sensory fibers are considered to play a major role to sense the bladder distention. However, in disease conditions of overactive bladder by, but not limited to, spinal cord injury, cystitis, Parkinson's disease, multiple sclerosis, previous cerebrovascular accident, and bladder outlet obstruction (BOO) caused by benign prostate hyperplasia (BPH), the sensitivity of C-fiber sensory neurons is upregulated and they contribute to the induction of the lower urinary tract symptoms. Treatment of overactive bladder patients with intravesical injection of capsaicin or its potent analog, resiniferatoxin, both of which desensitize VR1-positive C-fiber afferent neurons innervating the bladder, has been shown to be efficacious in several clinical trials (C. Silva et al, Eur Urol. 2000, 38(4), 444-452.). Therefore, C-fiber sensory neurons play an important role in the pathology of overactive bladder. PGI2 is generated locally in the bladder and it is the major prostaglandin released from the human bladder. In a rabbit BOO model, a stable metabolite of PGI2 was reported to be increased in BOO bladder (J M. Masick et al, Prostaglandins Other Lipid Mediat. 2001, 66(3), 211-219.).

Hence, PGI2 from disease bladder sensitizes C-fiber sensory neurons, and as a result, it may induce symptoms of overactive bladder. Therefore, IP receptor antagonists are expected to be useful in the treatment of overactive bladder and related urinary disorders.

WO 00/31045 discloses anti-thrombotics agents represented by the general formula:

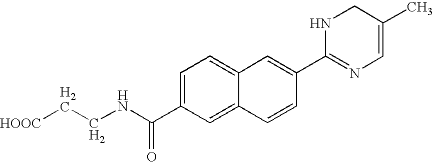

WO 98/44797 discloses integrin antagonists and farnesyl protein transferase inhibitors represented by the general formula:

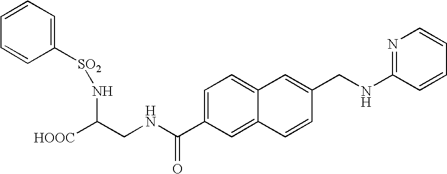

EP-A-220 118 discloses pharmaceutical composition intended for the treatment of dermatological, respiratory and ophthalmological conditions represented by the general formula:

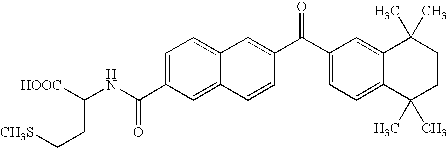

However, none of the references and other reference discloses 2-naphthamide derivatives having IP receptor antagonistic activity.

The development of a compound, which has effective IP receptor antagonistic activity and can be used for the prophylaxis and treatment of diseases alleviated by treatment with an IP receptor antagonist, has been desired.

### SUMMARY OF THE INVENTION

As the result of extensive studies on chemical modification of 2-naphthamide derivatives, the present inventors have found that the compounds of the structure related to the present invention have unexpectedly excellent IP receptor and/or antagonistic activity. The present invention has been accomplished based on these findings.

This invention is to provide a novel 2-naphthamide derivative of the formula (I), its tautomeric or stereoisomeric form, or a salt thereof:

(1) A 2-naphthamide derivative of the formula (1), its tautomeric or stereoisomeric form, or a salt thereof:

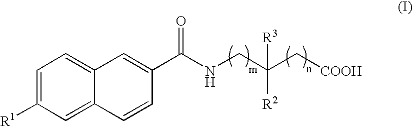

wherein

m and n independently represent an integer from 0 to 2;

—R¹ represents —O—R¹⁰—OR¹¹, —OR¹¹, —SR¹¹, —S(O)R¹¹, —S(O)₂R¹¹, —NR¹²R¹³, or —CHR¹⁴R¹⁵,
wherein
—R¹⁰— represents (C₁₋₆)alkylene;
R¹¹ represents aryl, (C₂₋₆)alkenyl optionally substituted by aryl or heteroaryl, (C₂₋₆)alkynyl optionally substituted by aryl or heteroaryl, or (C₁₋₆)alkyl optionally substituted by (C₃₋₈)-cycloalkyl, aryl or heterocycle comprising 4-9 carbons and at least one N, O, or S as a heteroatom,
   wherein
   said (C₃₋₈)cycloalkyl, aryl and heterocycle optionally have one or two substituents selected from the group consisting of halogen, hydroxy, nitro, (C₁₋₆)alkyl optionally substituted by mono-, di-, or tri halogen, and (C₁₋₆)alkoxy optionally substituted by (C₃₋₈)cycloalkyl, or mono-, di-, or tri halogen;
R¹² and R¹³ independently represent hydrogen, (C₂₋₆)alkenyl optionally substituted by aryl or heteroaryl, (C₂₋₆)alkynyl optionally substituted by aryl or heteroaryl, or (C₁₋₆)alkyl optionally substituted by aryl or heteroaryl,
or
R¹² and R¹³ form, together with the nitrogen atom, a 5-7 membered saturated hetero ring optionally interrupted by O or NH;
R¹⁴ and R¹⁵ independently represent hydrogen, (C₂₋₆)alkenyl optionally substituted by aryl or heteroaryl, (C₂₋₆)alkynyl optionally substituted by aryl or heteroaryl, (C₁₋₆)alkyl optionally substituted by aryl or heteroaryl, or (C₁₋₆)alkoxy optionally substituted by aryl or heteroaryl,
or
R¹⁴ and R¹⁵ form, together with the CH, a (C₃₋₈)cycloalkyl optionally interrupted by NH, or O, or a phenyl optionally substituted by hydroxy, halogen or (C₁₋₆)alkyl;

R² represents hydrogen, hydroxy, cyano, (C₁₋₆)alkoxy, (C₂₋₆)alkenyl, (C₂₋₆)alkynyl, (C₃₋₇)cycloalkyl, or (C₁₋₆)alkyl optionally having one or two substituents selected from the group consisting of hydroxy, amino, (C₁₋₆)alkylamino, aryl, and heteroaryl comprising 4-10 carbons and at least one N, O, or S as a heteroatom,
wherein
said aryl and heteroaryl optionally have one or two substituents selected from the group consisting of halogen, hydroxy, nitro, amino, N((C₁₋₆)alkyl sulfonyl)amino, morpholino, phenyl, pyridyl, (C₁₋₆)alkoxy optionally substituted by mono-, di-, or tri halogen, and (C₁₋₆)alkyl optionally substituted by mono-, di-, or tri halogen; and

R³ represents hydrogen, or (C₁₋₆)alkyl.

The compounds of the present invention show excellent IP receptor antagonistic activity. They are, therefore, suitable for the production of medicament or medical composition, which may be useful to treat IP receptor related diseases.

More specifically, since the 2-naphthamide derivatives of the present invention antagonize IP receptor, they are useful for treatment and prophylaxis of urological diseases or disorder.

Such diseases or disorders include bladder outlet obstruction, overactive bladder, urinary incontinence, detrusor hyper-reflexia, detrusor instability, reduced bladder capacity, frequency of micturition, urge incontinence, stress incontinence, bladder hyperreactivity, benigbn prostatic hypertrophy (BPH), prostatitis, urinary frequency, nocturia, urinary urgency, pelvic hypersensitivity, uretbritis, pelvic pain syndrome, prostatodynia, cystitis, or idiophatic bladder hypersensitivity.

The compounds of the present invention are also useful for treatment of pain including, but not limited to inflammatory pain, neuropathic pain, acute pain, chronic pain, dental pain, premenstrual pain, visceral pain, headaches, and the like; hypotension; hemophilia and hemorrhage; inflammation; respiratory states from allegies or asthma, since the disease is also alleviated by treatment with an IP receptor antagonist.

In another embodiment, the present invention provides 2-naphthamide derivatives of the formula (I′), its tautomeric or stereoisomeric form, or a salt thereof: 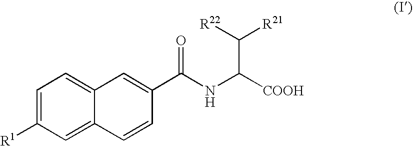
wherein
—R¹ represents —O—R¹—OR¹¹, —OR¹¹, —SR¹¹, —SOR¹¹, —SO₂R¹¹, —NR¹²R¹³, or —CHR¹⁴R¹⁵,
   wherein
   —R¹⁰— represents (C₁₋₆)alkylene;
R¹¹ represents aryl, (C₂₋₆)alkenyl optionally substituted by aryl or heteroaryl, (C₂₋₆)alkynyl optionally substituted by aryl or heteroaryl, or (C₁₋₆)alkyl optionally substituted by (C₃₋₈)-cycloalkyl, aryl or heterocycle comprising 4-9 carbons and at least one N, O, or S as a heteroatom
   wherein
   said (C₃₋₈)cycloalkyl, aryl and heterocycle optionally have one or two substituents selected from the group consisting of halogen, hydroxy, nitro, (C₁₋₆)alkyl optionally substituted by mono-, di-, or tri halogen, and (C₁₋₆)alkoxy optionally substituted by (C₃₋₈)cycloalkyl, or mono-, di-, or tri halogen;
   R¹² and R¹³ independently represent hydrogen, (C₂₋₆)alkenyl optionally substituted by aryl or heteroaryl, (C₂₋₆)alkynyl optionally substituted by aryl or heteroaryl, or (C₁₋₆)alkyl optionally substituted by aryl or heteroaryl,
   or
   R¹² and R¹³ form, together with the nitrogen atom, a 5-7 membered saturated hetero ring optionally interrupted by O or NH;
   R¹⁴ and R¹⁵ independently represent hydrogen, (C₂₋₆)alkenyl optionally substituted by aryl or heteroaryl, (C₂₋₆)alkynyl optionally substituted by aryl or heteroaryl, (C₁₋₆)alkyl optionally substituted by aryl or heteroaryl, or (C₁₋₆)alkoxy optionally substituted by aryl or heteroaryl,
   or
   R¹⁴ and R¹⁵ form, together with the CH, a (C₃₋₈)cycloalkyl optionally interrupted by NH, or O, or a phenyl optionally substituted by hydroxy, halogen or (C₁₋₆)alkyl;
R²¹ represents hydroxy, cyano, amino, (C₁₋₆)alkylamino, thienyl, pyridyl, naphthyl, 1H-pyrrolo[2,3-b]pyridin-3-yl, indolyl optionally substituted by halogen or hydroxy, or phenyl,
   wherein said phenyl and naphthyl optionally have one or two substituents selected from the group consisting of halogen, hydroxy, nitro, amino, N((C₁₋₆)alkyl)amino, di(C₁₋₆)alkylamino, N((C₁₋₆)alkyl sulfonyl)amino, morpholino, phenyl, pyridyl, (C₁₋₆)alkoxy optionally substituted by mono-, di-, or tri halogen, and (C₁₋₆)alkyl optionally substituted by mono-, di-, or tri halogen; and
R²² represents hydrogen or hydroxy.

Yet another embodiment of the compounds of formula (I) or (I′) are those wherein:
R¹ represents phenoxy, (C₁₋₆)alkoxy optionally substituted by cyclo-propyl, cyclohexyl, pyrrolidinyl, piperidinyl, imidazolyl, pyridyl, pyrrolyl, phenyl, or thiazolyl optionally substituted by (C₁₋₆)alkyl,
   wherein
   said phenyl has optionally one or two substituents selected from the group consisting of fluoro, chloro, bromo, nitro, hydroxy, (C₁₋₆)alkyl optionally substituted by mono-, di, or tri halogen, and (C₁₋₆)alkoxy optionally substituted by mono-, di, or tri halogen, cyclopropyl, or cyclohexyl.

Another embodiment of the compounds of formula (I) or (I′) are those wherein:
R¹ represents phenoxy(C₁₋₆)alkyl, phenoxy(C₁₋₆)alkenyl, phenoxy(C₁₋₆)-alkynyl, or phenyl(C₁₋₆)alkoxy.

Further embodiment of the compounds of formula (I) is those
wherein
R² represents phenyl (C₁₋₆)alkyl,
   wherein
   said phenyl has optionally one or two substituents selected from the group consisting of fluoro, chloro, bromo, iodo, hydroxy, nitro, amino, N(methanesulfonyl)amino, morpholino, phenyl, pyridyl, methoxy, ethoxy, and trifluoromethyl.

Further embodiment of the compounds of formula (I′) is those wherein:
R¹ represents phenoxy, (C₁₋₆)alkoxy optionally substituted by cyclo-propyl, cyclohexyl, pyrrolidinyl, piperidinyl, imidazolyl, pyridyl, pyrrolyl, phenyl, or thiazolyl optionally substituted by (C₁₋₆)alkyl,
   wherein
   said phenyl has optionally one or two substituents selected from the group consisting of fluoro, chloro, bromo, nitro, hydroxy, (C₁₋₆)alkyl optionally substituted by mono-, di, or tri halogen, and (C₁₋₆)alkoxy optionally substituted by mono-, di, or tri halogen, cyclopropyl, or cyclohexyl;
R²¹ represents cyano, thienyl, pyridyl, phenyl, naphthyl, 1H-pyrrolo[2,3-b]pyridin-3-yl, or indolyl optionally substituted by halogen or hydroxy,
   wherein
   said phenyl and naphthyl have one or two substituents selected from the group consisting of fluoro, chloro, bromo, hydroxy, nitro, amino, N((C₁₋₆)alkyl)amino, di(C₁₋₆)alkylamino, N((C₁₋₆)alkyl sulfonyl)-amino, morpholino, phenyl, pyridyl, trifluoromethyl, trifluoro-methyloxy, (C₁₋₆)alkoxy, and (C₁₋₆)alkyl; and
R²² represents hydrogen or hydroxy.

More preferably, said 2-naphthamide derivatives of the formula (I) or (I′) are selected-from the group consisting of:
N-[6-(benzyloxy)-2-naphthoyl]phenylalanine;
N-[6-(benzyloxy)-2-naphthoyl](trifluoromethyl)phenylalanine;
N-{6-[(4-fluorobenzyl)oxy]-2-naphthoyl}phenylalanine;
N-{6-[(3-fluorobenzyl)oxy]-2-naphthoyl}phenylalanine;
N-{6-[(2-fluorobenzyl)oxy]-2-naphthoyl}phenylalanine;
N-[6-(3-pyridinylmethoxy)-2-naphthoyl]phenylalanine;
N-{6-[(3,4-difluorobenzyl)oxy]-2-naphthoyl}phenylalanine;
N-{6-[2-(1H-pyrrol-1-yl)ethoxy]-2-naphthoyl}phenylalanine;
N-[6-(4-pyridinylmethoxy)-2-naphthoyl]phenylalanine;
N-[6-(benzyloxy)-2-naphthoyl]-3-(triuoromethyl)phenylalanine;
N-[6-(benzyloxy)-2-naphthoyl]tryptophan;
N-[6-(benzyloxy)-2-naphthoyl]-O-methyltyrosine;
N-[6-(benzyloxy)-2-naphthoyl]-3-methoxytyrosine;
N-[6-(benzyloxy)-2-naphthoyl]-β-hydroxyphenylalanine;
N-[6-(2-phenylethoxy)-2-naphthoyl]phenylalanine;
N-[6-(benzyloxy)-2-naphthoyl]-4-chlorophenylalanine;
N-[6-(benzyloxy)-2-naphthoyl]-3-fluorophenylalanine;
N-{6-[(2-chlorobenzyl)oxy]-2-naphthoyl}phenylalanine;
N-{6-[(3-chlorobenzyl)oxy]-2-naphthoyl}phenylalanine;
N-{6-[(2-methoxybenzyl)oxy]-2-naphthoyl}phenylalanine;
N-{6-[(3-methoxybenzyl)oxy]-2-naphthoyl}phenylalanine;
N-{6-[(2,3-dichlorobenzyl)oxy]-2-naphthoyl}phenylalanine;
N-{6-[(3,5-dichlorobenzyl)oxy]-2-naphthoyl}phenylalanine;
N-{6-[(3,5-diethoxybenzyl)oxy]-2-naphthoyl}phenylalanine;
N-[6-(benzyloxy)-2-naphthoyl]-3-(2-thienyl)alanine;
N-[6-(benzyloxy)-2-naphthoyl]-4-bromophenylalanine;
N-[6-(benzyloxy)-2-naphthoyl]-4-nitrophenylalanine;
N-[6-(benzyloxy)-2-naphthoyl]-3-hydroxyphenylalanine;
N-[6-(benzyloxy)-2-naphthoyl]-3-(1-naphthyl)alanine;
N-[6-(benzyloxy)-2-naphthoyl]-5-hydroxytryptophan;
N-[6-(benzyloxy)-2-naphthoyl]-2-fluorophenylalanine;
N-{6-[(2-bromobenzyl)oxy]-2-naphthoyl}phenylalanine;
N-{6-[(3-bromobenzyl)oxy]-2-naphthoyl}phenylalanine;
N-{6-[(2-methylbenzyl)oxy]-2-naphthoyl}phenylalanine;
N-{6-[(3-methylbenzyl)oxy]-2-naphthoyl}phenylalanine;
N-{6-[(3-nitrobenzyl)oxy]-2-naphthoyl}phenylalanine;
N-[6-(benzyloxy)-2-naphthoyl]-3-(2-naphthyl)alanine;
N-[6-(benzyloxy)-2-naphthoyl]-4-iodophenylalanine;
N-[6-(benzyloxy)-2-naphthoyl]-5-fluorotryptophan;
N-[6-(benzyloxy)-2-naphthoyl]-3-(1H-pyrrolo[2,3-b]pyridin-3-yl)alanine;
N-{6-[2-(4-pyridinyl)ethoxy]-2-naphthoyl}phenylalanine;
N-{6-[(3-ethoxybenzyl)oxy]-2-naphthoyl}phenylalanine; and
N-[6-(2-phenylpropoxy)-2-naphthoyl]phenylalanine;

Further, the present invention provides a medicament which include one of the compounds described above and optionally pharmaceutically acceptable excipient.

The Alkyl per se and “alk” and “alkyl” in alkoxy, alkylene, alkanoyl, alkylamino, alkylaminocarbonyl, alkylaminosulphonyl, alkylsulphonylamino, alkoxycarbonyl, alkoxycarbonylamino and alkanoylamino represent a linear or branched alkyl radical having generally 1 to 6, preferably 1 to 4 and particularly preferably 1 to 3 carbon atoms, representing illustratively and preferably methyl, ethyl, n-propyl, isopropyl, tert-butyl, n-pentyl and n-hexyl.

Alkoxy illustratively and preferably represents methoxy, ethoxy, n-propoxy, isopropoxy, tert-butoxy, n-pentoxy and n-hexoxy.

Alkylamino represents an alkylamino radical having one or two (independently selected)alkyl substituents, illustratively and preferably representing methylamino, ethylamino, n-propylamino, isopropylamino, tert-butylamino, n-pentylamino, n-hexyl-amino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

Aryl per se represents a mono- to tricyclic aromatic carbocyclic radical having generally 6 to 14 carbon atoms, illustratively and preferably representing phenyl, naphthyl and phenanthrenyl.

Heterocyclic ring per se and hetero ring in heteroaryl refers to a 3- to 15-membered ring radical which consists of carbon atoms and from one to five heteroatoms selected from the group consisting of nitrogen, oxygen and sulfur. The heterocyclic ring radical may be a monocyclic, bicyclic or tricyclic ring system, which may include fused or bridged ring systems; and the nitrogen, carbon or sulfur atoms in the heterocyclic ring radical may be optionally oxidized and the heterocyclic ring system may be partially or fully saturated or aromatic. Examples of such rings include, but are not limited to thienyl, furyl, benzothienyl, furanyl, benzofuranyl, pyrazinyl, pyrazolyl, pyridazinyl, pyridyl, pyrimidinyl, pyrrolyl, isothiazolyl, thiazolyl, oxazolyl, isoxazolyl, triazolyl, tetrazolyl, imidazolyl, thiadiazoyl, benzothiadiazolyl, oxadiazolyl, benzothiazolyl, indolyl, indazolyl, carbazolyl, quinolyl, isoqinolyl, benzodioxolyl, indazolyl, indazolinolyl, pyrrolidinyl, piperidinyl, pyranyl, pyrazolinyl, piperazinyl, morpholinyl, thiamorpholinyl, thiazolidinyl, benzofuranoyl, thiamorpholinyl sulfone, benzoxazolyl, oxopiperidinyl, oxopyrrolidinyl, oxoazopinyl, azepinyl, furazanyl, tetrahydropyranyl, tetrahydrofuranyl, dioxolyl, dioxinyl, oxathiolyl, benzodioxolyl and the like

### EMBODIMENT OF THE INVENTION

The compound of the formula (I) of the present invention can be, but not limited to be, prepared by combining various known methods. In some embodiments, one or more of the substituents, such as amino group, carboxyl group, and hydroxyl group of the compounds used as starting materials or intermediates are advantageously protected by a protecting group known to those skilled in the art. Examples of the protecting groups are described in “Protective Groups in Organic Synthesis (3rd Edition)” by Greene and Wuts, John Wiley and Sons, New York 1999.

The compound of the formula (I) of the present invention can be, but not limited to be, prepared by the method [A] below.

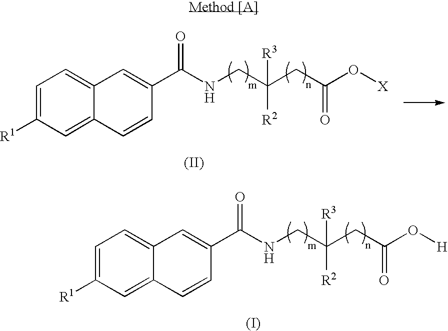

The compound of the formula (I) (wherein R¹, R², R³, m, and n are the same as defined above) can be prepared by deprotection of the compound of formula (II) (wherein R¹, R², R³, m, and n are the same as defined above and X represents C₁₋₆ alkyl, benzyl, 4-methoxybenzyl or 3,4-dimethoxybenzyl).

The deprotection of carboxyl group can be conducted by using a base including, for instance, an alkali metal alkoxide such as sodium methoxide, sodium ethoxide and potassium tert-butoxide; alkali metal hydroxide such as sodium hydroxide, lithium hydroxide and potassium hydroxide, or an acid including, for instance, HCl, HBr, trifluoroacetic acid and BBr₃.

The removal of protective group Z₁ can be conducted by using a base including, for instance, sodium hydroxide, lithium hydroxide and potassium hydroxide, or an acid including, for instance, HCl, HBr, trifluoroacetic acid and BBr₃.

The deprotection can also be done by hydrogenation using a catalyst including, for instance, palladium on carbon and palladium hydroxide, when Z₁ is benzyl, 4-methoxybenzyl or 3,4-dimethoxybenzyl.

Also, the deprotection can be done by using a reagent such as ceric ammonium nitrate (CAN) or 2,3-dichloro-5,6-dicyano-1,4-benzoquinone (DDQ), when Z₁ is 4-methoxybenzyl or 3,4-dimethoxybenzyl.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; amides such as N,N-dimethylformamide (DMF), N,N-dimethylacetamide and N-methylpyrrolidone; sulfoxides such as dimethylsulfoxide DMSO); alcohols such as methanol, ethanol, 1-propanol, isopropanol and tert-butanol; water, and others. Optionally, two or more of the solvents selected from the listed above can be mixed and used.

The reaction temperature can be optionally set depending on the compounds to be reacted. The reaction temperature is usually, but not limited to, about 20° C. to 100° C. The reaction may be conducted for, usually, 30 minutes to 48 hours and preferably 1 to 24 hours.

Preparation of Compound Formula (II)

Procedure A-I

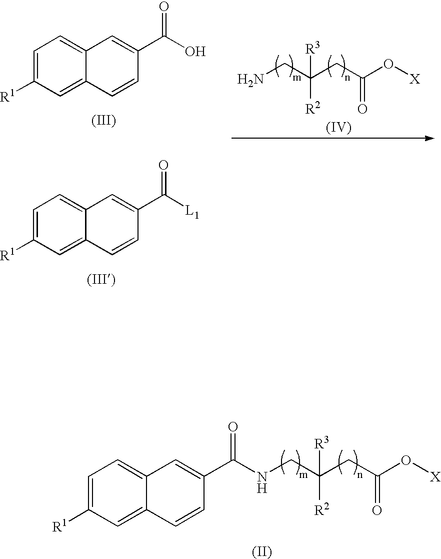

The compound of formula (II) (wherein R¹, R², R³, X, m and n are the same as defined above) can be prepared by the reaction of compound (III) (wherein R¹ is the same as defined above) or (III′) (wherein R¹ is the same as defined above and L₁ is leaving group, for instance, halogen atom such as chlorine, bromine, or iodine atom and azole such as 1,3-imidazole and 1,2,4-triazole) with the compound of formula (IV) (wherein R², R³, X, m and n are the same as defined above).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (TBF)and 1,2-dimethoxyethane; aromatic hydrocarbons such as benzene, toluene and xylene; amides such as N,N-dimethylformamide (DMF), N,N-dimethylacetamide and N-methylpyrrolidone; sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 0° C. to 100° C. The reaction may be conducted for, usually, 30 minutes to 48 hours and preferably 1 to 24 hours.

The reaction of compound of (III) and (IV) may be carried out using coupling agent including, for instance, carbodiimides such as N,N-dicyclohexylcarbodiimide and 1-(3-dimethylaminopropyl)-3-ethylcarbodiimide, 1-hydroxybenzotiazole, and others.

The reaction of compound of (III) and (IV) can also be advantageously conducted in the presence of a base, including, for instance, such as pyridine, triethylamine and N,N-diisopropylethylamine, dimethylaniline, diethylaniline, and others.

The compound (III), (III′) and amine (IV) can be commercially available or can be prepared by the use of known techniques.
Procedure A-II-a

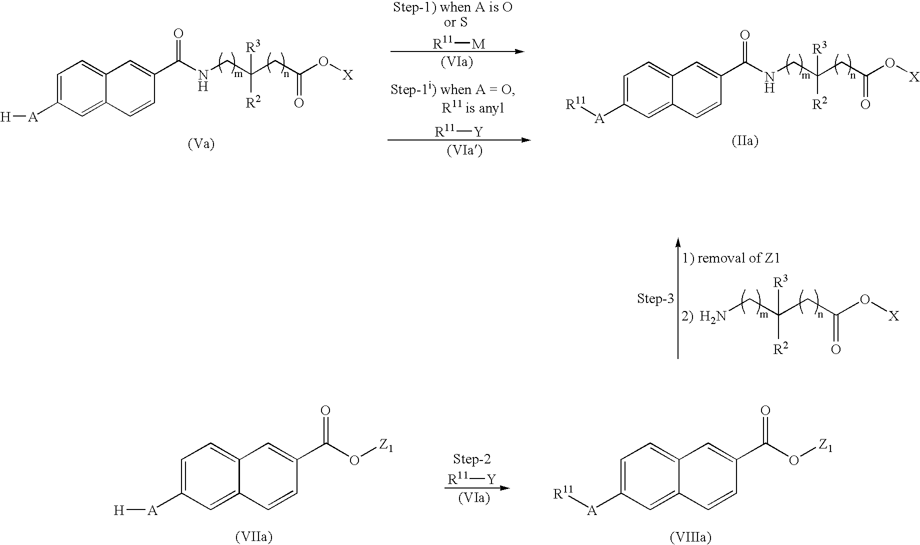

Step 1; the compound of formula (IIa) (wherein R², R³, R¹¹, X, m and n are the same as defined above and A represents O or S) can be prepared by the reaction of compound (Va) (wherein R², R³, X, m and n are the same as defined above and A represents O or S) with the compound of formula (VIa) (wherein R¹¹ is the same as defined above and Y represents a leaving group, e.g., halogen, and alkylsulfonyloxy).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF)and 1,2-diimethoxyethane; aromatic hydrocarbons such as benzene, toluene and xylene; amides such as N,N-dimethylformamide (DMF), N,N-dimethylacetamide and N-methylpyrrolidone; sulfoxides such as dimethylsulfoxide (DMSO); ketones such as acetone; alcohols such as methanol, ethanol, 1-propanol, isopropanol and tert-butanol; and others. Optionally, two or more of the solvents selected from the listed above can be mixed and used.

The reaction temperature can be optionally set depending on the compounds to be reacted. The reaction temperature is usually, but not limited to, about 0° C. to 100° C. The reaction may be conducted for, usually, 30 minutes to 48 hours and preferably 1 to 24 hours.

The reaction can be advantageously carried out in the presence of a base including, for instance, an alkali metal hydride such as sodium hydride or potassium hydride; alkali metal alkoxide such as sodium methoxide, sodium ethoxide and potassium tert-butoxide; alkali metal hydroxide such as sodium hydroxide and potassium hydroxide; alkali metal carbonates such as sodium carbonate and potassium carbonate; alkali metal hydrogen carbonates such as sodium hydrogen carbonate and potassium hydrogen carbonate; alkaline earth metal alkoxides such as magnesium ethoxide; organic amines such as pyridine, triethylamine and N,N-diisopropylethyl-amine, dimethylaniline, diethylaniline and others.

Step 1′; the compound of formula (IIa) (wherein R², R³, X, m and n are the same as defined above, A represents O and R¹¹ represents aryl) can be prepared by the reaction of compound (Va) (wherein R², R³, X, m and n are the same as defined above, A represents O and R¹¹ represents aryl) with the compound of formula (VIa′) (wherein R¹¹ represents aryl and M represents metal group including, for instance, organoborane group such as boronic acid and di-methoxy boryl; organostannyl group such as tributyl stannyl, and the like) in the presence of a copper catalyst such as copper(II) acetate and others.

The reaction can be advantageously carried out in the presence of a base including, for instance, cesium carbonate, sodium carbonate, potassium carbonate, pyridine, triethylamine and others.

The reaction may be carried out in a solvent including, for instance, ethers such as diethyl ether, isopropyl ether, dioxane and tetrahydrofuran (THF)and 1,2-dimethoxyethane; aromatic hydrocarbons such as benzene, toluene and xylene; amides such as N,N-dimethylformamide (DMF), N,N-dimethylacetamide and N-methyl-pyrrolidone; sulfoxides such as dimethylsulfoxide (DMSO); alcohols such as methanol, ethanol, 1-propanol, isopropanol and tert-butanol; and others.

Optionally, two or more of the solvents selected from the listed above can be mixed and used.

The reaction temperature can be optionally set depending on the compounds to be reacted. The reaction temperature is usually, but not limited to, about 0° C. to 120° C. The reaction may be conducted for, usually, 30 minutes to 48 hours and preferably 1 to 24 hours.

Step 2; the compound of formula (VIIIa) (wherein R¹¹ is the same as defined above, A represents O or S and Z₁ represents C₁₋₆ alkyl, benzyl, 4-methoxybenzyl, 3,4-dimethoxybenzyl and others) can be prepared by the reaction of compound (VIIa) (wherein Z₁ is the same as defined above and A represents O or S) with the compound of formula (VIa) (wherein R¹¹ is the same as defined above and Y represents a leaving group, e.g., halogen, and alkylsulfonyloxy) in the similar manner described in the step 1 of Procedure A-II-a, for the preparation of the compound of formula (IIa).

Step 3; the compound of formula (IIa) (wherein R², R³, R¹¹, X, m and n are the same as defined above and A represents O or S) can be prepared by 1) the removal of protective group Z₁ of the compound of the formula (VIIIa) and then 2) the reaction with the compound of the formula (V) (wherein R², R³, X, m and n are the same as defined above).

The removal of protective group Z₁ can be done in the similar manner described in the Method A for the preparation of the compound of formula (I).

The successive reaction with the compound of the formula (IV) (wherein R², R³, X, m and n are the same as defined above) can be carried out in the similar manner described in Procedure A-I, for the preparation of the compound of formula (II).

Procedure A-II-b

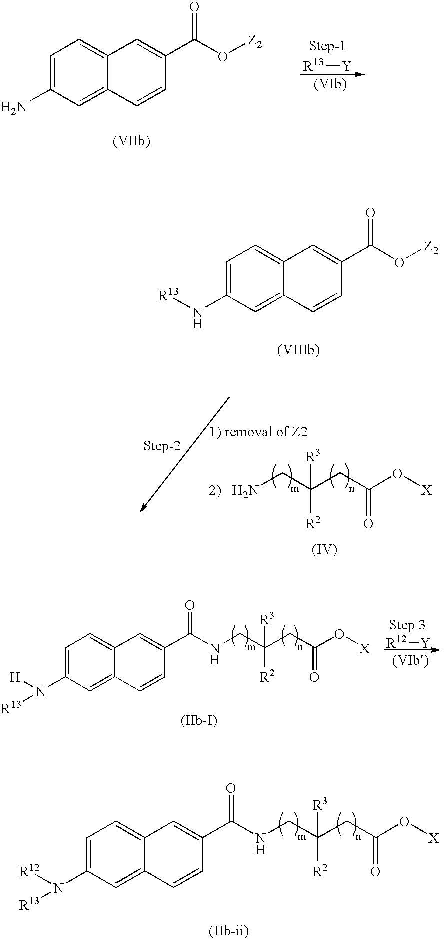

Step 1; the compound of formula (VIIIb) (wherein R¹³ is the same as defined above and Z₂ represents C₁₋₆ alkyl, benzyl, 4-methoxybenzyl, 3,4-dimethoxybenzyl and others ) can be prepared by the reaction of compound (VIIb) (wherein Z₂ is the same as defined above) with the compound of formula (VIb) (wherein R¹³ is the same as defined above and Y represents a leaving group, e.g., halogen, and alkylsulfonyloxy).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF)and 1,2-dimethoxyethane; aromatic hydrocarbons such as benzene, toluene and xylene; amides such as N,N-dimethylformamide (DMF), N,N-dimethylacetamide and N-methylpyrrolidone; sulfoxides such as dimethylsulfoxide (DMSO); ketones such as acetone; alcohols such as methanol, ethanol, 1-propanol, isopropanol and tert-butanol; and others. Optionally, two or more of the solvents selected from the listed above can be mixed and used.

The reaction temperature can be optionally set depending on the compounds to be reacted. The reaction temperature is usually, but not limited to, about 20° C. to 180° C. The reaction may be conducted for, usually, 30 minutes to 48 hours and preferably 2 to 24 hours.

The reaction can be advantageously carried out in the presence of a base including, for instance, cesium carbonate, sodium carbonate, potassium carbonate, pyridine, triethylamine and others.

Step 2; the compound of formula (IIb-i) (wherein R², R³, R¹³, X, m and n are the same as defined above) can be prepared by 1) the removal of protective group Z₂ of the compound of the formula (VIIIb) and then 2) the reaction with the compound of the formula (IV) (wherein R², R³, X, m and n are the same as defined above) in the similar manner described in Procedure A-I, for the preparation of the compound of formula (II).

Step 3, the compound of formula (IIb-ii) (wherein R², R³, R¹², R¹³, X, m and n are the same as defined above) can be prepared by the reaction of compound (IIb-i) (wherein R², R³, R¹³, X, m and n are the same as defined above) with the compound of formula (VIb′) (wherein R¹² is the same as defined above and Y represents a leaving group, e.g., halogen and alkylsulfonyloxy) in the similar manner described in step 1 of Procedure A-II-a, for the preparation of the compound of formula (II-a).

The compound (Va) can be commercially available or can be prepared by either the use of the similar procedure for the preparation of the compound of formula (II) or known techniques. The compound (VIa), (VIa′), (VIIa), (VIb), (Vib′) and (VIIb) can be commercially available or can be prepared by the use of known techniques.

When the compound shown by the formula (I) or a salt thereof has an asymmetric carbon in the structure, their optically active compounds and racemic mixtures are also included in the scope of the present invention.

Typical salts of the compound shown by the formula (I) include salts prepared by reaction of the compounds of the present invention with a mineral or organic acid, or an organic or inorganic base. Such salts are known as acid addition and base addition salts, successively.

Acids to form salts include inorganic acids such as, without limitation, sulfuric acid, phosphoric acid, hydrochloric acid, hydrobromic acid, hydriodic acid and the like, and organic acids, such as, without limitation, p-toluenesulfonic acid, methane-sulfonic acid, oxalic acid, p-bromophenylsulfonic acid, succinic acid, citric acid, benzoic acid, acetic acid, and the like.

Base addition salts include those derived from inorganic bases, such as, without limitation, ammonium hydroxide, alkaline metal hydroxide, alkaline earth metal hydroxides, carbonates, bicarbonates, and the like, and organic bases, such as, without limitation, ethanolamine, triethylamine, tris(hydroxymethyl)aminomethane, and the like. Examples of inorganic bases include, sodium hydroxide, potassium hydroxide, potassium carbonate, sodium carbonate, sodium bicarbonate, potassium bicarbonate, calcium hydroxide, calcium carbonate, and the like.

The compound of the present invention or a salts thereof, depending on its substituents, may be modified to form lower alkylesters or known other esters; and/or hydrates or other solvates. Those esters, hydrates, and solvates are included in the scope of the present invention.

The compound of the present invention may be administered in oral forms, such as, without limitation normal and enteric coated tablets, capsules, pills, powders, granules, elixirs, tinctures, solution, suspensions, syrups, solid and liquid aerosols and emulsions. They may also be administered in parenteral forms, such as, without limitation, intravenous, intraperitoneal, subcutaneous, intramuscular, and the like forms, well-known to those of ordinary skill in the pharmaceutical arts. The compounds of the present invention can be administered in intranasal form via topical use of suitable intranasal vehicles, or via transdermal routes, using transdermal delivery systems well-known to those of ordinary skilled in the art.

The dosage regimen with the use of the compounds of the present invention is selected by one of ordinary skill in the arts, in view of a variety of factors, including, without limitation, age, weight, sex, and medical condition of the recipient, the severity of the condition to be treated, the route of administration, the level of metabolic and excretory function of the recipient, the dosage form employed, the particular compound and salt thereof employed.

The compounds of the present invention are preferably formulated prior to administration together with one or more pharmaceutically-acceptable excipients. Excipients are inert substances such as, without limitation carriers, diluents, flavoring agents, sweeteners, lubricants, solubilizers, suspending agents, binders, tablet disintegrating agents and encapsulating material.

Yet another embodiment of the present invention is pharmaceutical formulation comprising a compound of the invention and one or more pharmaceutically-acceptable excipients that are compatible with the other ingredients of the formulation and not deleterious to the recipient thereof. Pharmaceutical formulations of the invention are prepared by combining a therapeutically effective amount of the compounds of the invention together with one or more pharmaceutically-acceptable excipients. In making the compositions of the present invention, the active ingredient may be mixed with a diluent, or enclosed within a carrier, which may be in the form of a capsule, sachet, paper, or other container. The carrier may serve as a diluent, which may be solid, semi-solid, or liquid material which acts as a vehicle, or can be in the form of tablets, pills, powders, lozenges, elixirs, suspensions, emulsions, solutions, syrups, aerosols, ointments, containing, for example, up to 10% by weight of the active compound, soft and hard gelatin capsules, suppositories, sterile injectable solutions and sterile packaged powders.

For oral administration, the active ingredient may be combined with an oral, and non-toxic, pharmaceutically-acceptable carrier, such as, without limitation, lactose, starch, sucrose, glucose, sodium carbonate, mannitol, sorbitol, calcium carbonate, calcium phosphate, calcium sulfate, methyl cellulose, and the like; together with, optionally, disintegrating agents, such as, without limitation, maize, starch, methyl cellulose, agar bentonite, xanthan gun, alginic acid, and the like; and optionally, binding agents, for example, without limitation, gelatin, natural sugars, beta-lactose, corn sweeteners, natural and synthetic gums, acacia, tragacanth, sodium alginate, carboxymethylcellulose, polyethylene glycol, waxes, and the like; and, optionally, lubricating agents, for example, without limitation, magnesium stearate, sodium stearate, stearic acid, sodium oleate, sodium benzoate, sodium acetate, sodium chloride, talc, and the like.

In powder forms, the carrier may be a finely divided solid which is in admixture with the finely divided active ingredient. The active ingredient may be mixed with a carrier having binding properties in suitable proportions and compacted in the shape and size desired to produce tablets. The powders and tablets preferably contain from about 1 to about 99 weight percent of the active ingredient which is the novel composition of the present invention. Suitable solid carriers are magnesium carboxymethyl cellulose, low melting waxes, and cocoa butter.

Sterile liquid formulations include suspensions, emulsions, syrups and elixirs. The active ingredient can be dissolved or suspended in a pharmaceutically acceptable carrier, such as sterile water, sterile organic solvent, or a mixture of both sterile water and sterile organic solvent.

The active ingredient can also be dissolved in a suitable organic solvent, for example, aqueous propylene glycol. Other compositions can be made by dispersing the finely divided active ingredient in aqueous starch or sodium carboxymethyl cellulose solution or in a suitable oil.

The formulation may be in unit dosage form, which is a physically discrete unit containing a unit dose, suitable for administration in human or other mammals. A unit dosage form can be a capsule or tablets, or a number of capsules or tablets. A “unit dose” is a predetermined quantity of the active compound of the present invention, calculated to produce the desired therapeutic effect, in association with one or more excipients. The quantity of active ingredient in a unit dose may be varied or adjusted from about 0.1 to about 1000 milligrams or more according to the particular treatment involved.

Typical oral dosages of the present invention, when used for the indicated effects, will range from about 0.01 mg/kg/day to about 100 mg/kg/day, preferably from 0.1 mg/kg/day to 30 mg/kg/day, and most preferably from about 0.5 mg/kg/day to about 10 mg/kg/day. In the case of parenteral administration, it has generally proven advantageous to administer quantities of about 0.001 to 100 mg/kg/day, preferably from 0.01 mg/kg/day to 1 mg/kg/day. The compounds of the present invention may be administered in a single daily dose, or the total daily dose may be administered in divided doses, two, three, or more times per day. Where delivery is via transdermal forms, of course, administration is continuous.

### EXAMPLES

The present invention will be described in detail below in the form of examples, but they should by no means be construed as defining the metes and bounds of the present invention.

In the examples below, all quantitative data, if not stated otherwise, relate to percentages by weight.

Melting points are uncorrected. Liquid Chromatography-Mass spectroscopy (LC-MS) data were recorded on a Micromass Platform LC with Shimadzu Phenomenex ODS column (4.6 mm×30 mm) flushing a mixture of acetonitrile-water (9:1 to 1:9) at 1 ml/min of the flow rate. Mass spectra were obtained using electrospray (ES) ionization techniques (micromass Platform LC). TLC was performed on a precoated silica gel plate (Merck silica gel 60 F-254). Silica gel (WAKO-gel C-200 (75-150 μm)) was used for all column chromatography separations. All chemicals were reagent grade and were purchased from Sigma-Aldrich, Wako pure chemical industries, Ltd., Great Britain, Tokyo kasei kogyo Co., Ltd., Japan, Nacalai tesque, Inc., Watanabe Chemical Ind. Ltd., Maybridge plc, Lancaster Synthesis Ltd., Great Britain, Merck KgaA, Germany, Kanto Chemical Co., Ltd.

The effect of the present compounds were examined by the following assays and pharmacological tests.

[Measurement of the [³H]-iloprost Binding to HEL Cells] (Assay 1)

A human erythioleukemia cell line, HEL 92.1.7, was purchased from American Type Culture Correction and maintained in RPMI-1640 medium (Gibco BRL) supplemented with 10% fetal calf serum (FCS), 2 mM glutamine, 4.5 g/L glucose, 10 mM Hepes, 1 mM sodium pyruvate, 100 U/ml penicillin, and 100 μg/ml streptomycin in a humidified 5% CO₂ atmosphere at 37° C. Cells were collected with centrifugation and washed with binding assay buffer (BAB: 50 mM Tris-HCl, 5 mM MgCl₂ (pH 7.5)). Cells were suspended at the density of 6.25×10⁶ cells/ml in BAB, and one million cells in 160 μl aliquot of cell suspension were put in a well of 96 well plate Falcon). Then, 20 μl of compound solution, 100 μM of iloprost (for non-specific binding), or buffer alone (total binding), diluted with 1% DMSO in BAB was added. Finally, another 20 μl containing [³ H]-iloprost (0.02 μCi, 0.5-1 pmol) in BAB was added and incubated at room temperature for 30 min with a gentle shaking. Cell suspension was then transferred to a well of MultiScreen plate with GF/C glass filters (Millipore) to harvest cells. Cells were washed twice with 200 μl of ice-cold BAB and the plate was kept at 55° C. for 30 min to dry filters. The filter in the well was punched out to a counting tube and 2 ml of Ultima Gold XR (Packard) was added. [³ H]-radio activity in the filter was measured by a liquid scintillation counter (Beckman, USA).

[Iloprost-Induced cAMP Production Assay in HEL Cells] (Assay 2)

HEL cells were collected with centrifugation and washed with cAMP assay buffer (CAB: Hank's balanced salt solution, 17 mM Hepes, 0.1% bovine serum albumin, 1 mM IBMX, 0.4% DMSO, and 1 mM L-ascorbic acid sodium salt (pH 7.4)). Cells were suspended at the density of 2.5×10⁵ cells/ml in CAB, and twenty thousand cells in 80 μl aliquot of cell suspension were put in a well of 96 well plate (Falcon). Then, 10 μl of compound solution diluted with 1% DMSO in CAB or buffer alone was added. The plate was incubated at 37° C. for 30 min. Then, another 10 μl containing 100 nM iloprost in CAB or buffer alone was added and further incubated at 37° C. for 30 min. cAMP content in the well was measured by a cAMP ELISA kit (Applied Biosystems, USA).

[Measurement of Rhythmic Bladder Contraction in Anesthetized Rats]

(1) Animals

Female Sprague-Dawley rats (200˜250 g/Charles River Japan) were used.

(2) Rhythmic Bladder Contraction in Anesthetized Rats

Rats were anesthetized by intraperitoneal administration of urethane (Sigma) at 1.25 g/kg. The trachea was cannulated with a polyethylene tube (HIBIKI, No. 8) to facilitate respiration; and a cannula (BECTON DICKINSON, PE-50) was placed in the left femoral vein for intravenous administration of testing compounds. The abdomen was opened through a midline incision, and after both ureters were cut, a water-filled baloon (about 1 ml capacity) was inserted through the apex of the bladder dome. The balloon was connected to a pressure transducer onto a polygraph. Rhythmic bladder contraction was elicited by raising up intravesical pressure to approximately 15 cm H₂O. After the rhythmic bladder contraction was stable, a testing compound was administered intravenously. Activity was estimated by measuring disappearance time and amplitude of the rhythmic bladder contraction. The effect on amplitute of bladder contractions was expressed as a percent suppression of the amplitude of those after the disappearance was recovered. Experimental values were expressed as the mean±S.E.M. The testing compounds-mediated inhibition of the rhythmic bladder contraction was evaluated using Student's t-test. A probability level less than 5% was accepted as significant difference.

Results of Iloprost-induced cAMP production assay (Assay2) are shown in Examples and tables of the Examples below. For practical reasons, the compounds are grouped in three classes of activity as follows:
A(=or<)0.1μM<B(=or<)1μM<C

The compounds of the present invention also show excellent selectivity, and strong activity in vivo assays.

### Example 1-1

### (1) N-(6-Hydroxy-2-naphthoyl)phenylalanine Methyl Ester

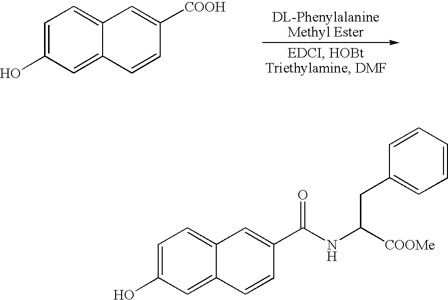

To a solution of 6-hydroxy-2-naphthoic acid (300 mg), DL-phenylalanine methyl ester (344 mg), 1-hydroxybenzotriazole (HOBt, 280 mg), and triethylamine (0.3 ml) in N,N-dimethylformamide (DMF, 8 ml) was added 1-ethyl-3-(3-dimethylamino-propyl)carbodiimide (EDCI, 396 mg). The mixture was stirred at room temperature overnight and concentrated in vacuo. The residue was extracted with ethyl acetate and washed with brine. The organic layer was dried over sodium sulfate and evaporated to give colorless viscous oil, that was purified by silica gel column chromatography (hexane/ethyl acetate=1:1) to give N-(6-hydroxy-2-naphthoyl)phenylalanine methyl ester (475 mg, 85%) as a colorless foam.

### (2) N-(6-Benzyloxy-2-naphthoyl)phenylalanine Methyl Ester

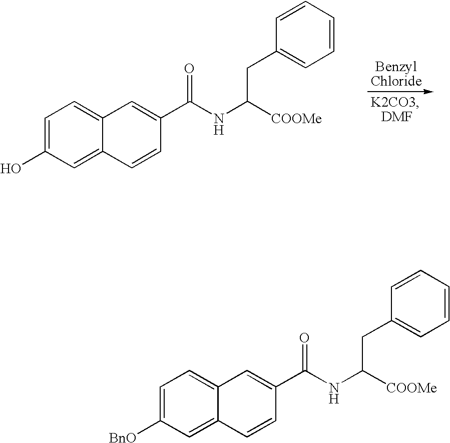

To a solution of N-(6-hydroxy-2-naphthoyl)phenylalanine methyl ester (200 mg) and benzyl chloride (80 μl) in DMF (5 ml) was added potassium carbonate (95 mg). The mixture was stirred at room temperature overnight and at 70° C. for 2 hours. The solvent was evaporated in vacuo and the residue was extracted with ethyl acetate and washed with brine. The organic layer was dried over sodium sulfate and concentrated in vacuo to give a colorless solid, that was purified by preparative TLC (chloroform/ethyl acetate=10/1) and crystallization with diisopropyl ether to give N-(6-benzyloxy-2-naphthoyl)phenylalanine methyl ester (190 mg, 76%) as a colorless solid.

### (3) N-(6-Benzyloxy-2-naphthoyl)phenylalanine

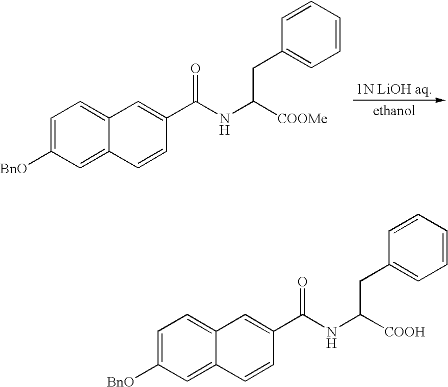

To a solution of N-(6-benzyloxy-2-naphthoyl)phenylalanine methyl ester (60 mg) in ethanol (2 ml) was added 1N lithium hydroxide water solution (0.2 ml). The mixture was stirred at 50° C. for 4 hours and concentrated in vacuo. The residue was diluted with water and acidified with 1N hydrochloric acid to be extracted with a mixture of acetate and tetrahydroflarane. The organic layer was washed with brine and dried over sodium sulfate. The solvent was evaporated off to give a colorless oil, that was crystallized with diisopropylether to give N-(6-benzyloxy-2-naphthoyl)phenylalanine (47.8 mg, 82%) as a colorless solid.

Melting point: 230° C.

Molecular weight: 425.48

Mass spectrometry: 426

Activity grade assay 2: A

¹H-NMR (500 MHz, DMSO-d6): δ 3.11 (1H, dd, J=13.9, 10.4 Hz), 3.22 (1H, dd, J=13.8, 4.4 Hz), 4.64-4.69 (1H, m), 5.25 (2H, s), 7.17 (1H, t, J=7.2 Hz), 7.25-7.35 (6H, m), 7.42 (2H, t, J=7.2 Hz), 7.48 (1H, d, J=2.3 Hz), 7.52 (2H, d, J=7.3 Hz), 7.84 (2H, d, J=0.9 Hz), 7.93 (1H, d, J=9.1 Hz), 8.34 (1H, s), 8.73 (1H, d, J=8.2 Hz), 12.76 (1H, bs).

In the similar manner as described in Example 1-1, compounds in Example 1-2 to 1-94 as shown in Table 1 were synthesized.

TABLE 1

| | | | MASS | | |
|---|---|---|---|---|---|
| Ex No | Structure | Mol weight | (M + 1) | MP | Assay 2 |
| 1-2 | 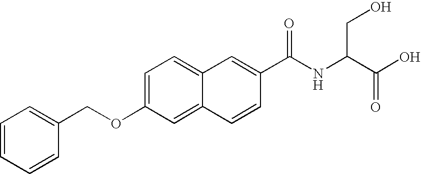 | 365.39 | 366 | 196-198 | C |
| 1-3 | 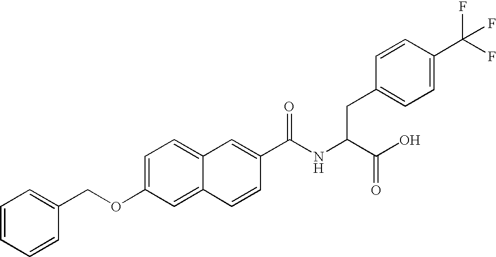 | 493.49 | 494 | 212-214 (dec.) | A |
| 1-4 | 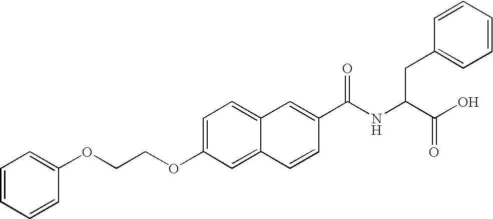 | 455.52 | 456 | 225-227 | B |
| 1-5 | 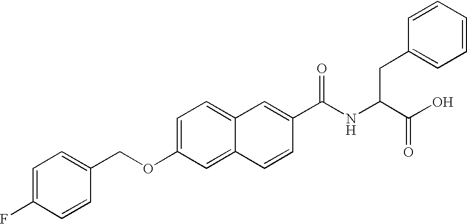 | 443.48 | 444 | 197 | A |
| 1-6 | 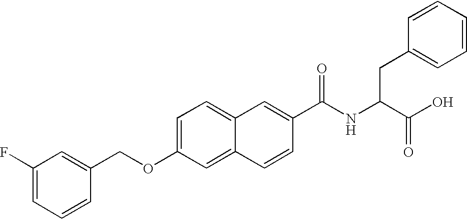 | 443.48 | 444 | 197-199 | A |
| 1-7 | 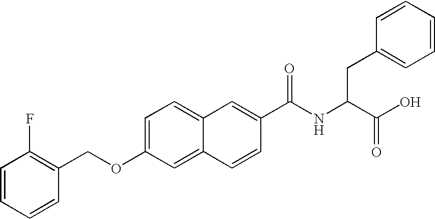 | 443.48 | 444 | 180-182 | A |
| 1-8 | 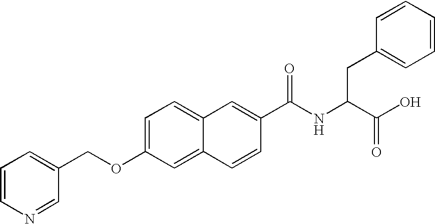 | 426.48 | 427 | 217 | A |
| 1-9 | 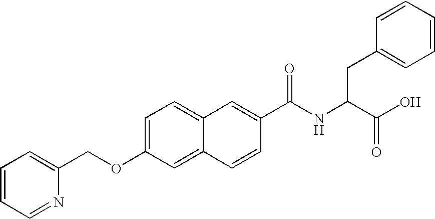 | 426.48 | 427 | 214 | B |
| 1-10 | 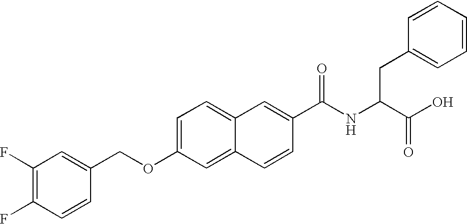 | 461.47 | 462 | 185 | A |
| 1-11 | 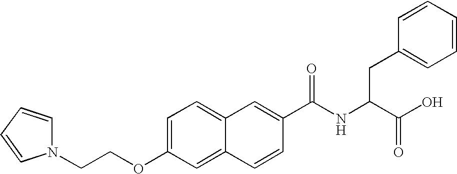 | 428.49 | 429 | 168-169 | A |
| 1-12 | 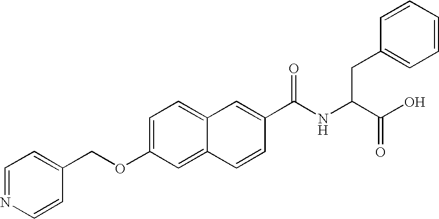 | 426.48 | 427 | 220-221 | A |
| 1-13 | 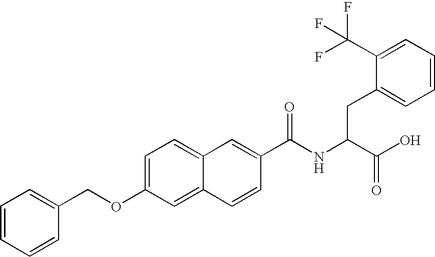 | 493.49 | 494 | ND | B |
| 1-14 | 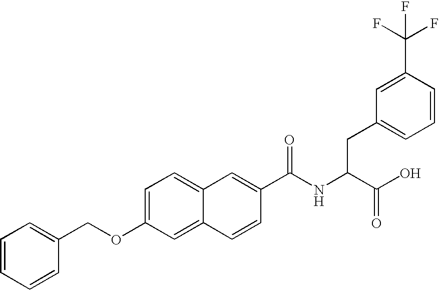 | 493.49 | 494 | ND | A |
| 1-15 | 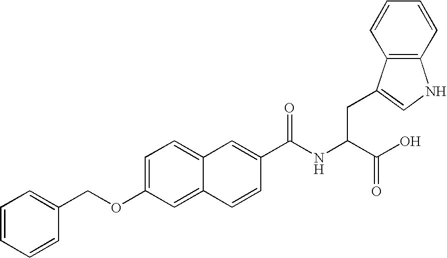 | 464.53 | 465 | ND | A |
| 1-16 | 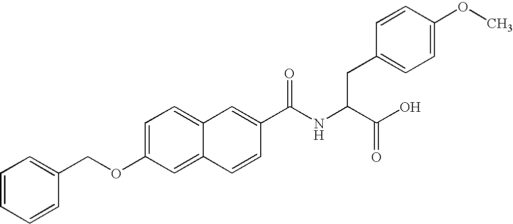 | 455.52 | 456 | ND | A |
| 1-17 | 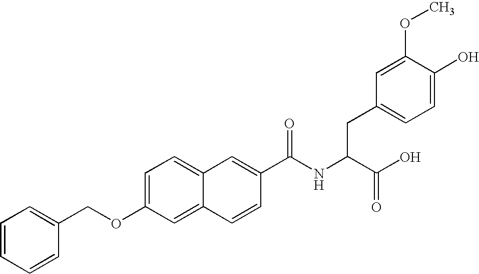 | 471.51 | 472 | ND | A |
| 1-18 | 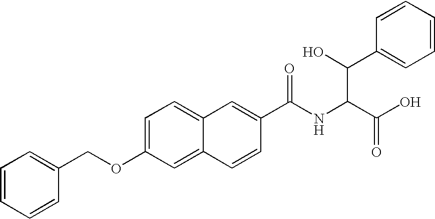 | 441.49 | 442 | ND | A |
| 1-19 | 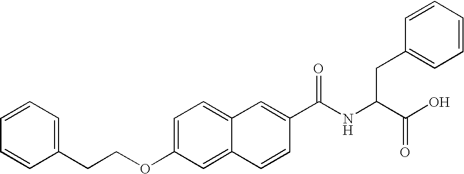 | 439.52 | 440 | 144 | A |
| 1-20 | 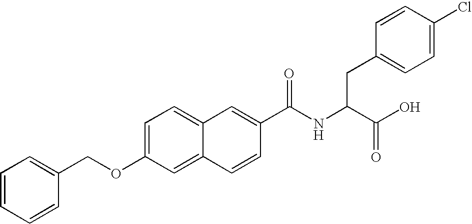 | 459.93 | 460 | 202-204 | A |
| 1-21 | 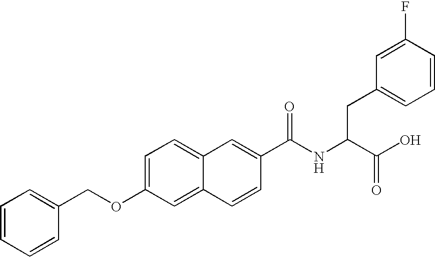 | 443.48 | 444 | 188-191 | A |
| 1-22 | 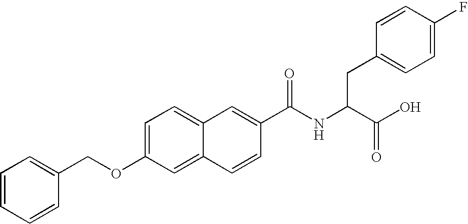 | 443.48 | 444 | 214-216 | A |
| 1-23 | 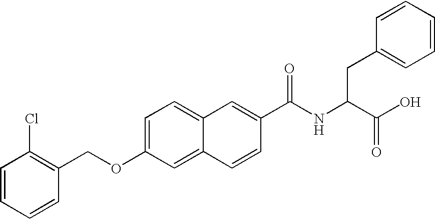 | 459.93 | 460 | 215 | A |
| 1-24 | 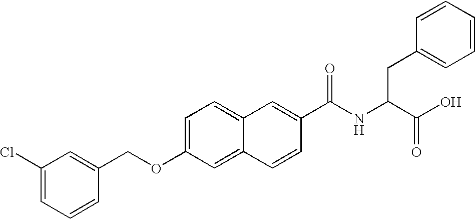 | 459.93 | 460 | 187 | A |
| 1-25 | 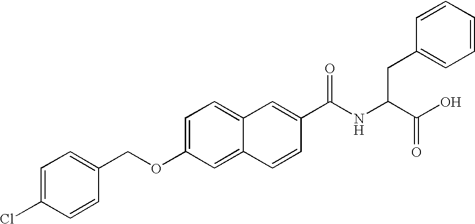 | 459.93 | 460 | 226 | B |
| 1-26 | 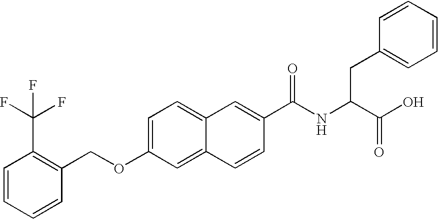 | 493.49 | 494 | 167 | B |
| 1-27 | 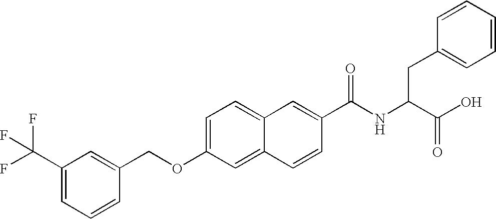 | 493.49 | 494 | 158 | C |
| 1-28 | 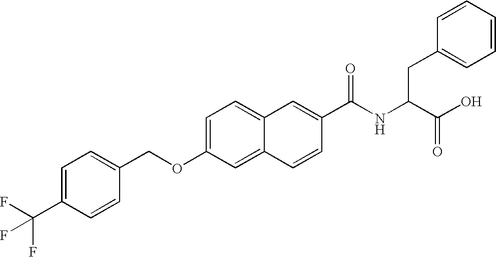 | 493.49 | 494 | 224 | C |
| 1-29 | 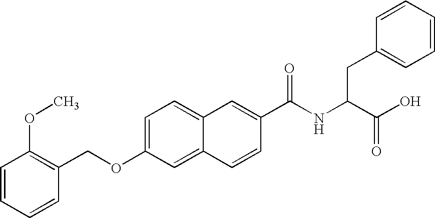 | 455.52 | 456 | 169 | A |
| 1-30 | 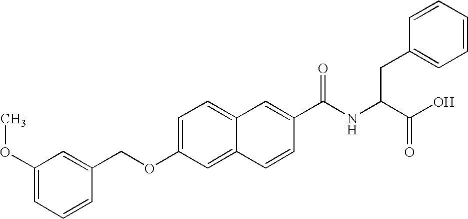 | 455.52 | 456 | 163 | A |
| 1-31 | 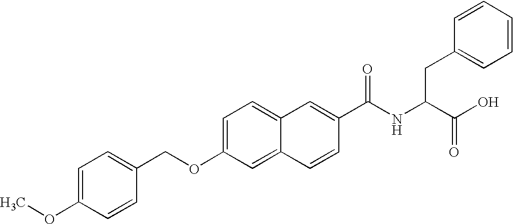 | 455.52 | 456 | 188 | C |
| 1-32 | 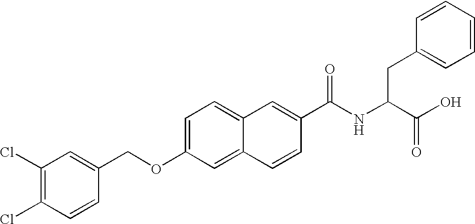 | 494.38 | 494 (M) | 202 | B |
| 1-33 | 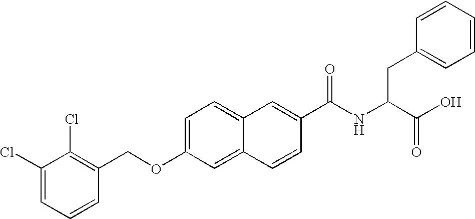 | 494.38 | 494 (M) | 211 | A |
| 1-34 | 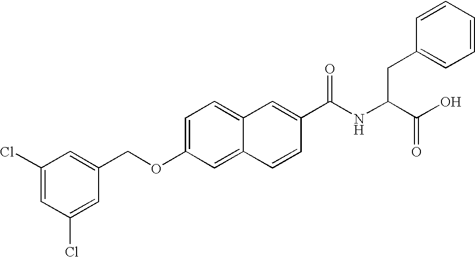 | 494.38 | 494 (M) | 180 | A |
| 1-35 | 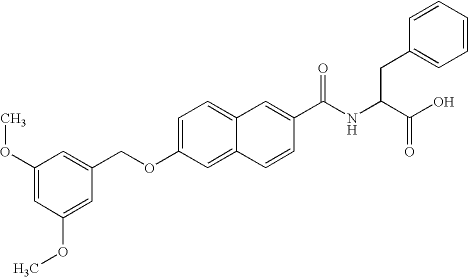 | 485.54 | 486 | 140 | A |
| 1-36 | 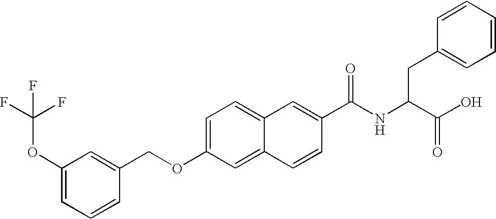 | 509.49 | 510 | 157 | B |
| 1-37 | 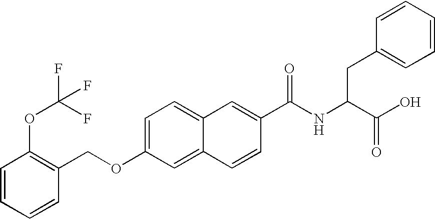 | 509.49 | 510 | 158 | C |
| 1-38 | 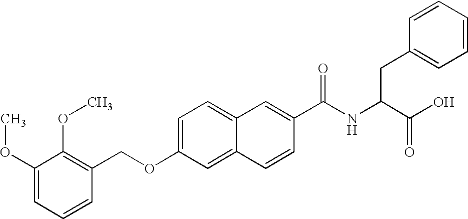 | 485.54 | 486 | 161 | C |
| 1-39 | 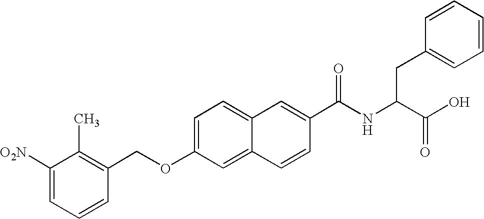 | 484.51 | 485 | 202 | B |
| 1-40 | 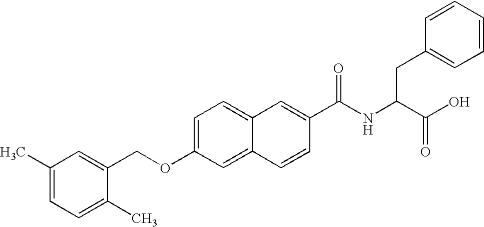 | 453.54 | 454 | 210 | B |
| 1-41 | 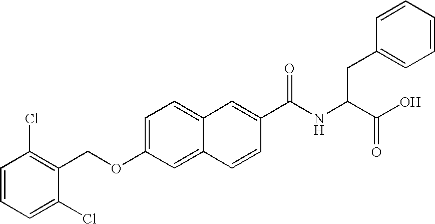 | 494.38 | 494 (M) | 245 | B |
| 1-42 | 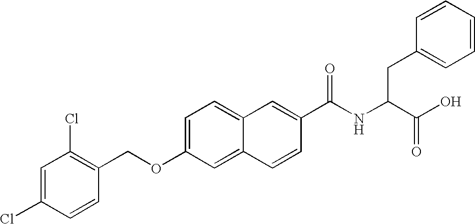 | 494.38 | 494 (M) | 202 | C |
| 1-43 | 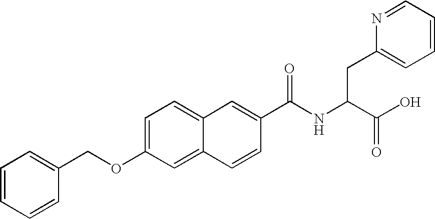 | 426.48 | 427 | 172-173 (dec.) | B |
| 1-44 | 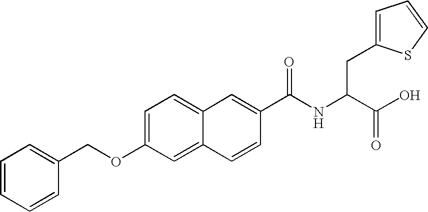 | 431.51 | 432 | 200-202 | A |
| 1-45 | 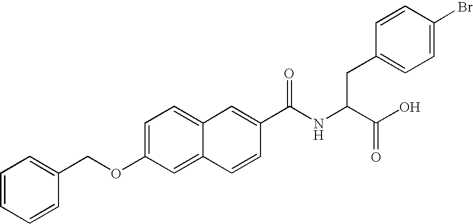 | 504.38 | 505 | 219-220 | A |
| 1-46 | 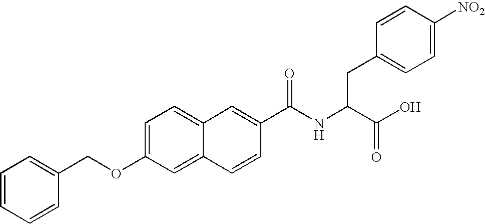 | 470.49 | 469 (M − 1) | 227-228 | A |
| 1-47 | 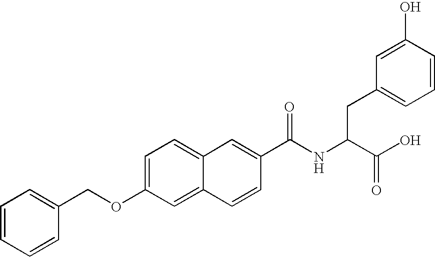 | 441.49 | 442 | 214 | A |
| 1-48 | 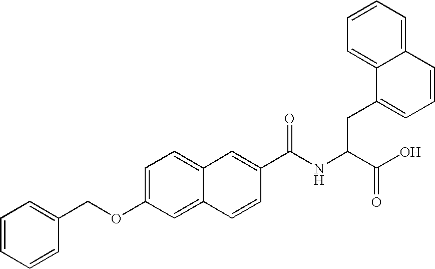 | 475.55 | 476 | 218 | A |
| 1-49 | 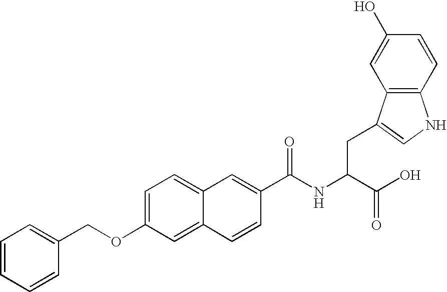 | 480.53 | 481 | 261-262 (dec.) | A |
| 1-50 | 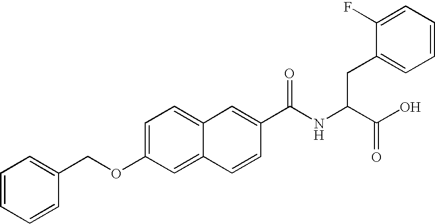 | 443.48 | 444 | 211-213 | A |
| 1-51 | 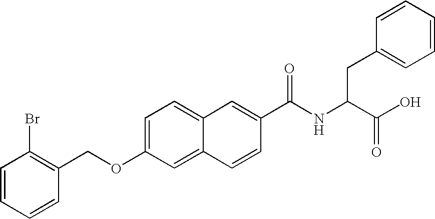 | 504.38 | 504 (M) | 206-207 | A |
| 1-52 | 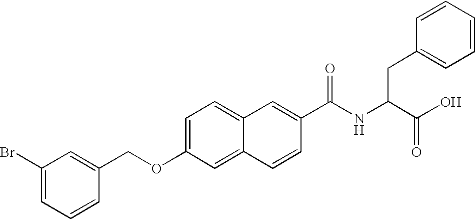 | 504.38 | 504 | 194 | A |
| 1-53 | 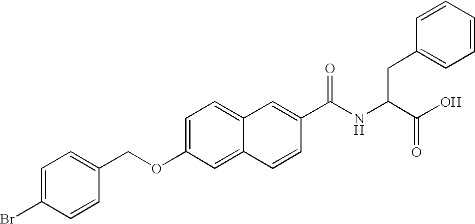 | 504.38 | 504 | 232 | C |
| 1-54 | 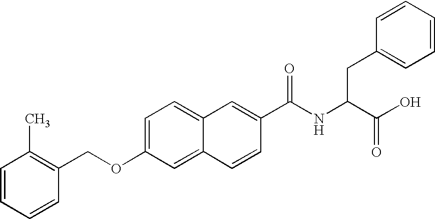 | 439.52 | 440 | 204 | A |
| 1-55 | 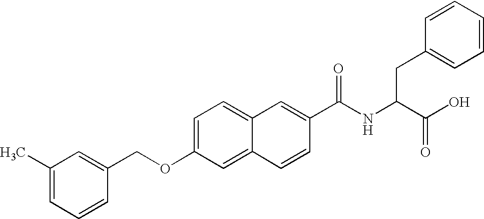 | 439.52 | 440 | 184-185 | A |
| 1-56 | 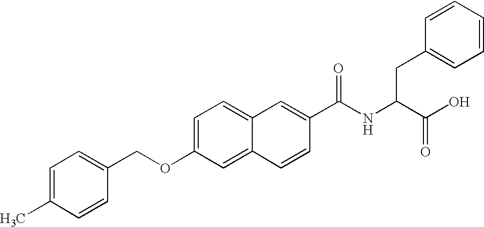 | 439.52 | 440 | 212 | B |
| 1-57 | 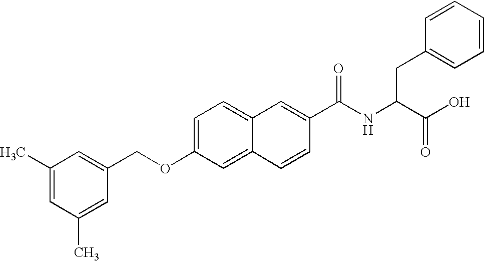 | 453.54 | 454 | 167-168 | B |
| 1-58 | 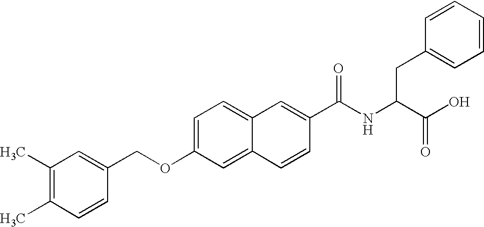 | 453.54 | 454 | 189 | B |
| 1-59 | 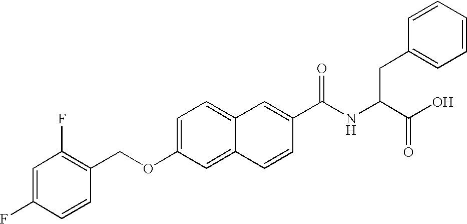 | 461.47 | 462 | 199 | B |
| 1-60 | 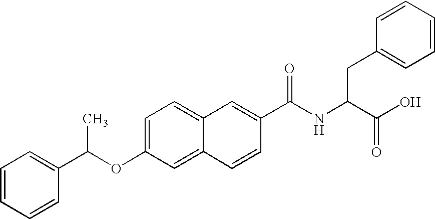 | 439.52 | 440 | 170-172 | B |
| 1-61 | 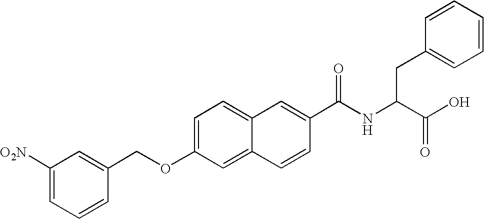 | 470.49 | 488 | 193-194 | A |
| 1-62 | 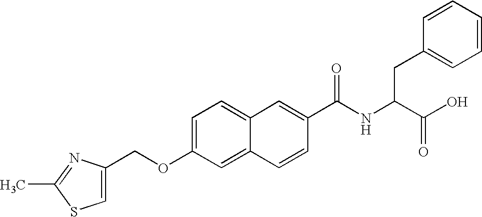 | 446.53 | 447 | 208 | B |
| 1-63 | 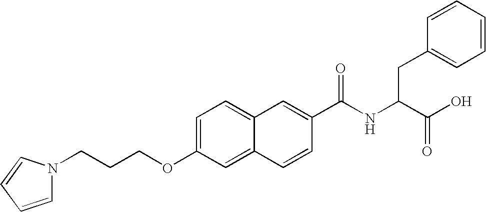 | 442.52 | 443 | 195 | B |
| 1-64 | 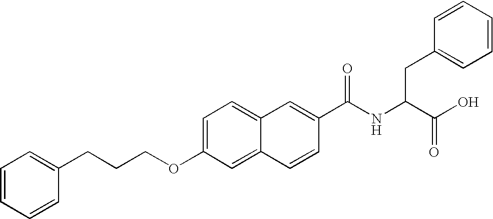 | 453.54 | 454 | 182 | B |
| 1-65 | 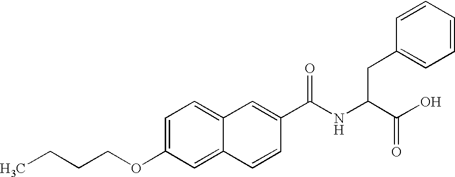 | 391.47 | 392 | 209 | B |
| 1-66 | 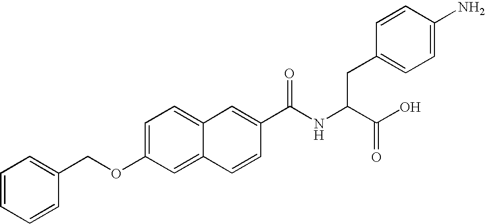 | 440.50 | 441 | 238-240 | C |
| 1-67 | 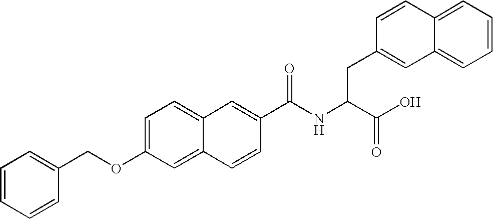 | 475.55 | 476 | 226-227 | A |
| 1-68 | 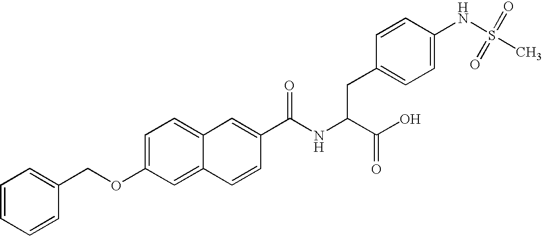 | 518.59 | 519 | 236-237 | C |
| 1-69 | 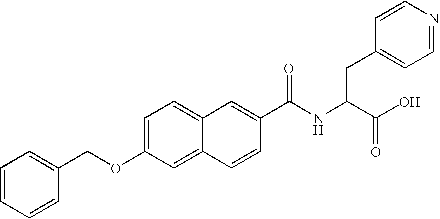 | 426.48 | 427 | 260-262 (dec.) | B |
| 1-70 | 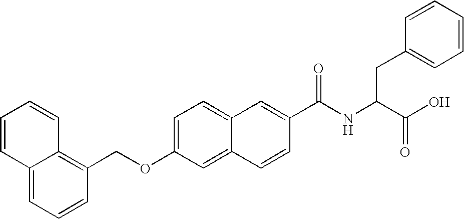 | 475.55 | 476 | 220 | B |
| 1-71 | 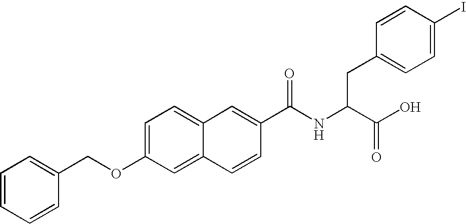 | 551.39 | 552 | 219-221 | A |
| 1-72 | 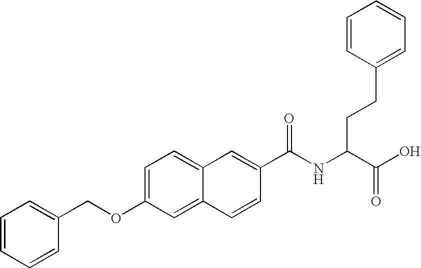 | 439.52 | 440 | 223-225 | C |
| 1-73 | 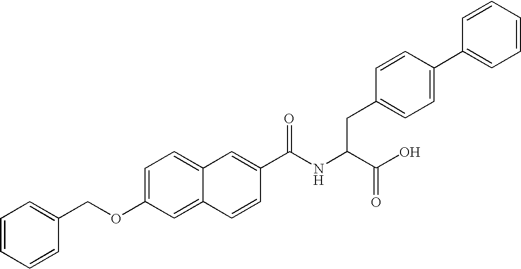 | 501.59 | 502 | 250-252 | B |
| 1-74 | 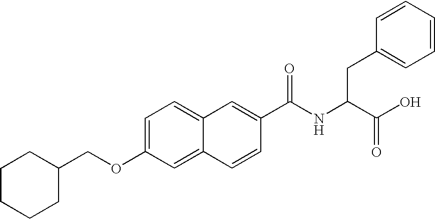 | 431.54 | 432 | 220 | B |
| 1-75 | 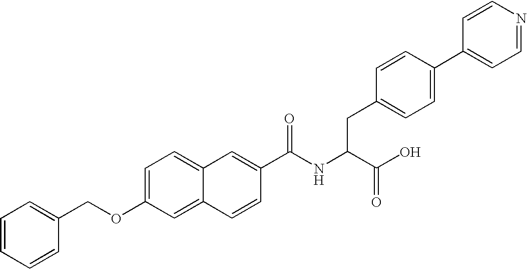 | 502.58 | 503 | 247-249 (dec.) | B |
| 1-76 | 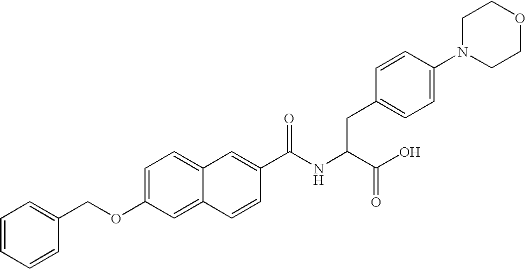 | 510.60 | 511 | 216-218 (dec.) | C |
| 1-77 | 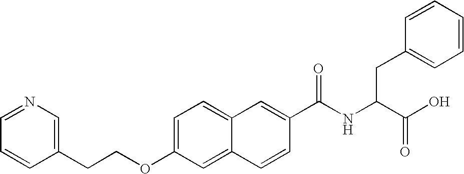 | 440.50 | 441 | 113 | B |
| 1-78 | 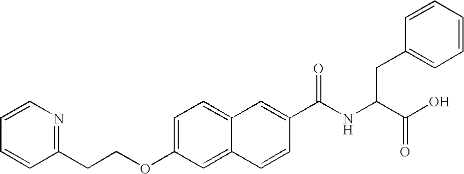 | 440.50 | 441 | 143 | B |
| 1-79 | 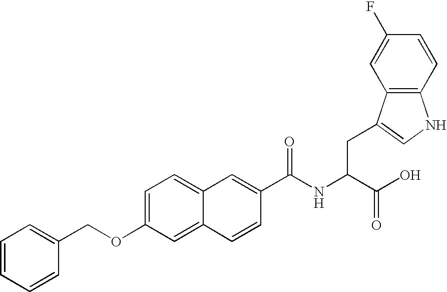 | 482.52 | 483 | 236-238 (dec.) | A |
| 1-80 | 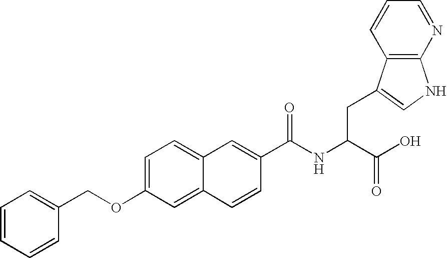 | 465.51 | 466 | 275-277 (dec.) | A |
| 1-81 | 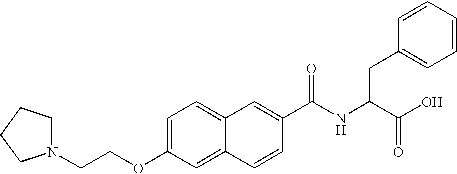 | 432.52 | 433 | 234 | C |
| 1-82 | 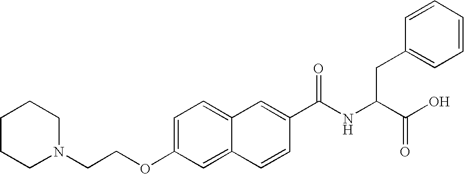 | 446.55 | 447 | 215 | C |
| 1-83 | 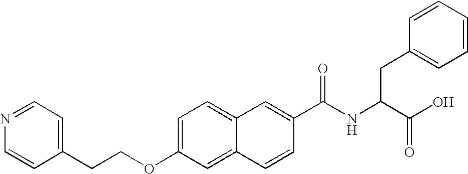 | 440.50 | 441 | 190 | A |
| 1-84 | 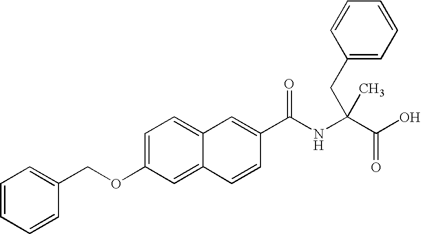 | 439.52 | 440 | 184 | B |
| 1-85 | 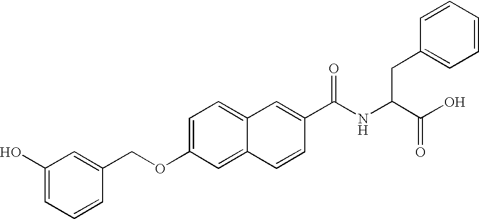 | 441.49 | 442 | 157 | A |
| 1-86 | 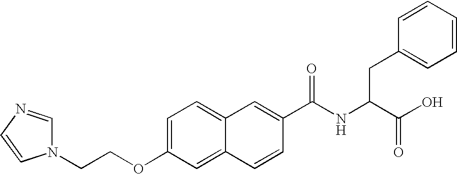 | 429.48 | 430 | 172 (dec.) | B |
| 1-87 | 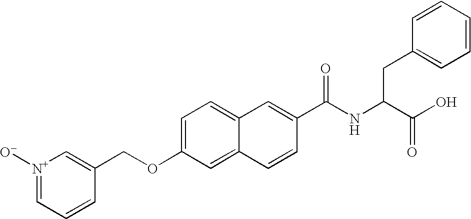 | 442.48 | 443 | 252 (dec.) | C |
| 1-88 | 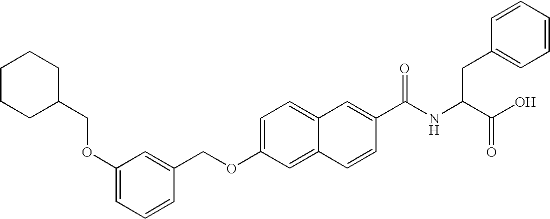 | 537.66 | 538 | 180 | C |
| 1-89 | 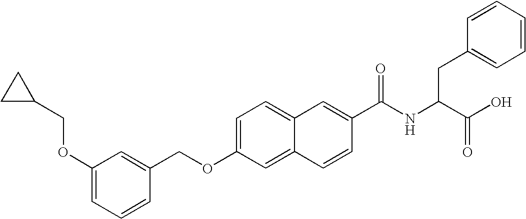 | 495.58 | 496 | 179 | B |
| 1-90 | 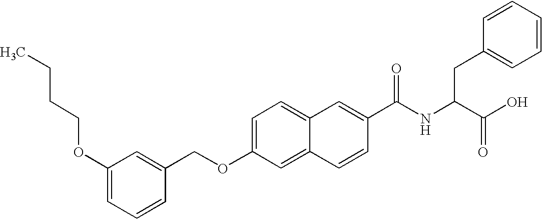 | 497.60 | 498 | 172 | B |
| 1-91 | 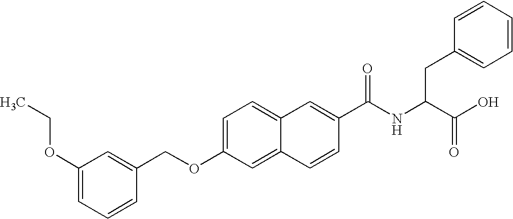 | 469.54 | 470 | 145 | A |
| 1-92 | 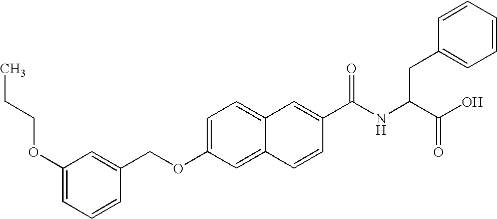 | 483.57 | 484 | 173 | B |
| 1-93 | 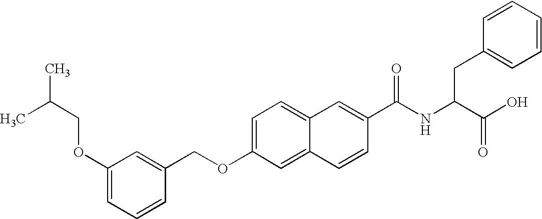 | 497.60 | 498 | 140 | B |
| 1-94 | 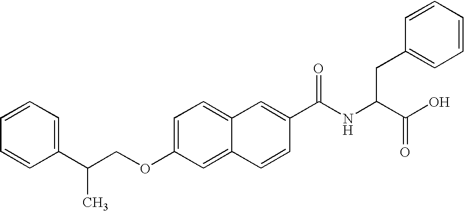 | 453.54 | 454 | 160 | A |

### Example 2-1

### (1) Benzyl 6-(benzyloxy)-2-naphthoate

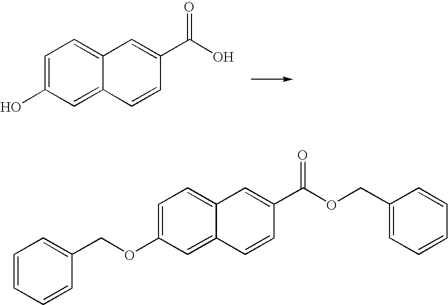

To a solution of 6-hydroxy-2-naphthoic acid (0.50 g, 2.66 mmol) and benzylchloride (1.01 g, 7.97 mmol) in N,N-dinethylformamide (15 mL) was added potassium carbonate (1.10 g, 7.97 mmol) and sodium iodide (0.12 g, 0.80 mmol), and the mixture was stirred at 70° C. for 4 hours. The mixture was concentrated in vacuo, and the residue was partitioned between ethyl acetate and water. The separated organic phase was dried over sodium sulfate, filtered and concentrated in vacuo. The residue was purified by column chromatography on silica-gel (n-hexane:ethyl acetate, 4:1) to give benzyl 6-(benzyloxy)-2-naphthoate (1.01 g, 103%) as yellowish granules.

### (2) 6-(Benzyloxy)-2-naphthoic Acid

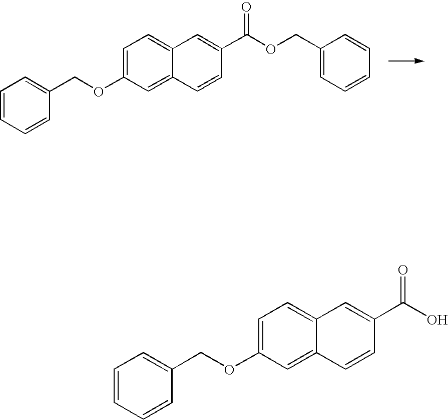

To a solution of benzyl (6-benzyloxy)-2-naphthoate (1.01 g, 2.75 mmol) in ethanol (20 ml) was added dropwise 1N NaOH (5.50 ml, 5.50 mmol) and the mixture was stirred at room temperature for 3 days. The mixture was concentrated under reduced pressure. The residue was diluted with water (10 ml) and washed with ether. The aqueous layer was separated and neutralized with 1N HCl (5.50 ml). The resultant precipitate was collected by filtration, washed successively with water and ethyl acetate and dried under reduced pressure to give 6-(benzyloxy)-2-naphthoic acid (0.42 g, 55%) as colorless powder.

### (3) tert-Butyl N-[6-(benzyloxy)-2-naphthoyl]-D-phenylalaninate

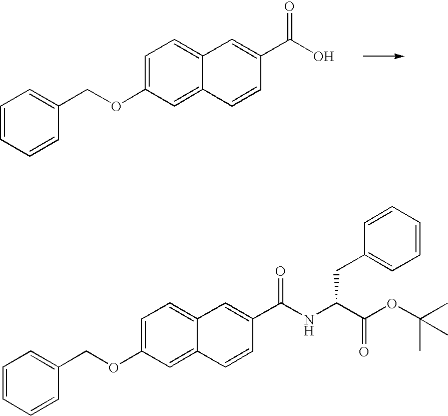

To a solution of 6-(benzyloxy)-2-naphthoic acid (200 mg, 0.72 mmol), D-phenyl-alanine tert-butyl ester hydrochloride salt(185 mg, 0.72 mmol), 1-hydroxybenzo-triazole (280 mg, 0.93 mmol) and triethylamine (0.30 ml, 0.86 mmol) in N,N-dimethylformamide (8.0 ml) was added 1-ethyl-3-(3-dimethylaminopropyl)-carbodiimide (396 mg, 0.93 mmol). The mixture was stirred at room temperature overnight and concentrated in vacuo. The residue was extracted with ethyl acetate and washed with brine. The organic layer was dried over sodium sulfate and evaporated to give colorless viscous oil, that was purified by silica gel column chromatography (hexane:ethyl acetate, 5:1) to give tert-butyl N-[6-(benzyloxy)-2-naphthoyl]-D-phenylalaninate (318 mg, 92%) as a colorless solid.

### (4) N-[6-(benzyloxy)-2-naphthoyl]-D-phenylalanine

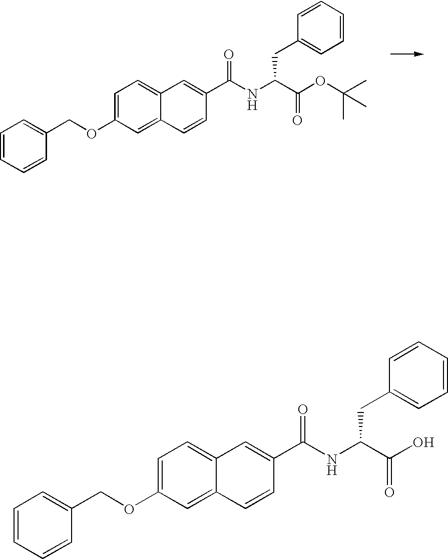 tert-Butyl N-[6-(benzyloxy)-2-naphthoyl]-D-phenylalaninate (100 mg, 0.21 mmol) was dissolved in 4N hydrochloride-dioxane (2.0 ml) and the resulting mixture was kept at room temperature overnight. The mixture was concentrated in vacuo. The residue was triturated with diisopropyl ether to give powder, which was recrystallized from a mixture of methylenechloride and diisopropyl ether to give N-[6-(benzyloxy)-2-naphthoyl]-D-phenylalanine (62 mg, 70%) as colorless powder.

Melting point: 179° C.

Molecular weight: 425.48

Mass spectrometry: 426

Activity grade assay 2: A

HPLC analysis
Column: Daicel Chiralcel OD-RH 5 μm 0.46 cm×15 cm
Eluent: 0.1% acetic acid in water:acetonitrile=50:50
Flow rate: 1.0 ml/min.
Absorbance: 210 nm
Retention time: 12.29 min. (N-[6-(benzyloxy)-2-naphthoyl]-D-phenylalanine)

### Example 2-2

### N-[6-(benzyloxy)-2-naphthoyl]-L-phenylalanine

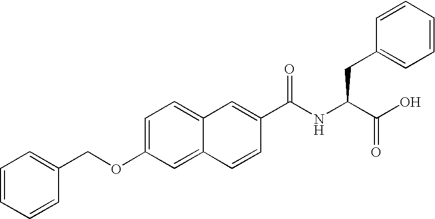

According to the similar synthetic procedure as in example 2-1, N-[6-(benzyloxy)-2-naphthoyl]-L-phenylalanine was obtained as a colorless powder.

Melting point: 207° C.

Molecular weight: 425.48

Mass spectrometry: 426

Activity grade assay2: C

HPLC analysis
Column: Daicel Chiralcel OD-RH 5 μm 0.46 cm×15 cm
Eluent: 0.1% acetic acid in water:acetonitrile=50:50
Flow rate: 1.0 ml/min.
Absorbance: 210 nm
Retention time: 11.10 min. (N-[6-(benzyloxy)-2-naphthoyl]-L-phenylalanine)

### Example 3-1

### N-(6-phenoxy-2-naphthoyl)phenylalanine

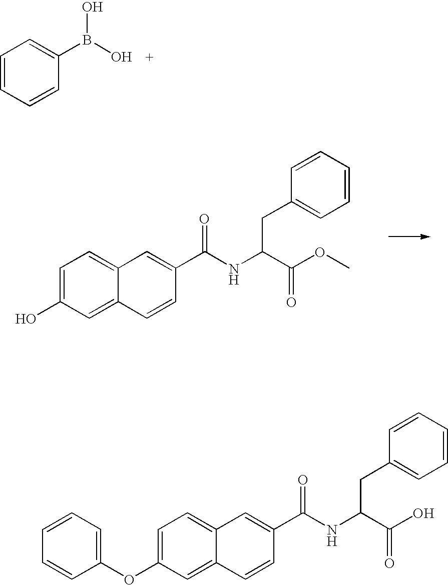

To a mixture of phenylboronic acid (0.141 g, 1.14 mmol), methyl N-(6-hydroxy-2-naphthoyl)phenylalaninate (0.200 g, 0.57 mmol), copper(II) acetate (0.104 g, 0.57 mmol) and molecular sieves 4A (570 mg) in dichloromethane was added triethylamine (0.290 g, 2.86 mmol), and stiring was continued at room temperature overnight. The reaction mixture was filtered and concentrated under reduced pressure. The crude product was purified by preparative TLC (hexane:ethyl acetate, 3:1) to give methyl N-(6-phenoxy-2-naphthoyl)phenylalaninate as a colorless oil.

The methyl ester above was treated with lithium hydroxide monohydrate (0.036 g, 0.86 mmol) in tetrahydrofuran (1.0 mL), ethanol (0.5 mL) and water (1.0 mL) at 60° C. for 2 hours. The mixture was neutralized with 1N hydrochloric acid and extracted with ethyl acetate. The separated organic phase was washed with brine, dried over sodium sulfate, filtered and concentrated under reduced pressure. The residue was purified by recrystallization (ethyl acetate-hexane) to give N-(6-phenoxy-2-naphthoyl)phenylalanine (0.020 g, 9%) as a white powder.

Melting point: 179-180° C.

Molecular weight: 411.46

Mass spectrometry: 412

Activity grade assay 2: C

¹H-NMR (500 MHz, DMSO-d6): δ 3.11(1H, dd, J=10.4, 13.9 Hz), 3.22 (1H, dd, J=4.7, 13.9 Hz), 4.66 (1H, br), 7.10-7.29 (6H, m), 7.30-7.38 (4H, m), 7.46 (2H, m), 7.85 (2H, t, J=8.8 Hz), 8.06 (1H, d, J=8.8 Hz), 8.40 (1H, s), 8.77 (1H, br), 12.78 (1H, br).

### Example 4-1

### (1) Benzyl 6-(benzylamino)-2-naphthoate

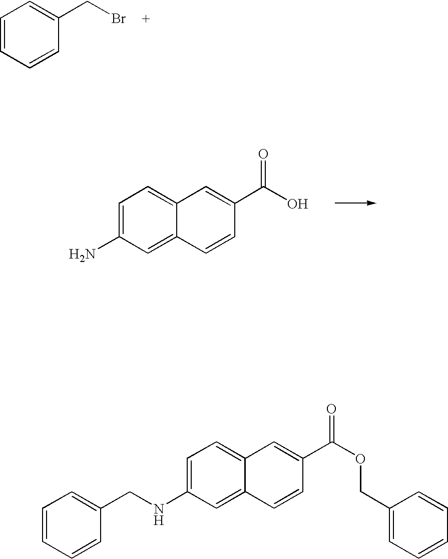

To a solution (40 mL) of 6-amino-2-naphtoic acid (0.500 g, 2.67 mmol) in DMF were added benzyl bromide (1.142 g, 6.68 mmol), potassium carbonate (1.107 g, 8.01 mmol) and sodium iodide (1.201 g, 8.01 mmol), and the mixture was stirred at 80° C. overnight. After cooled to room temperature, the mixture was partitioned between ethyl acetate and saturated aqueous ammonium chloride solution. The separated organic phase was washed with water and brine, dried over sodium sulfate, filtered and concentrated under reduced pressure. The crude product was purified by column chromatography on silica gel (exane:ethyl acetate, 3:1) to give benzyl 6-(benzylamino)-2-naphthoate (0.214 g, 21%) as a white solid.

### (2) 6-(benzylamino)-2-naphthoic acid

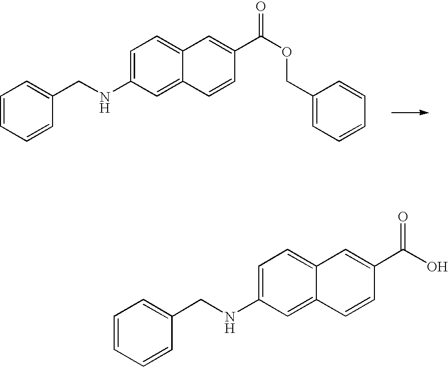

To a solution of benzyl 6-(benzylamino)-2-naphtoate (0.200 g, 0.54 mmol) in tetra-hydrofuran (2 mL), ethanol (1 mL) and water (2 mL) was added lithium hydroxide monohydrate (0.068 g, 1.63 mmol), and the mixture was stirred at 60° C. overnight. After cooled to room temperature, the mixture was partitioned between ethyl acetate and saturated ammonium chloride aqueous solution. The separated organic phase was washed with water and brine, dried over sodium sulfate, filtered and concentrated under reduced pressure to give 6-benzylamino-2-naphtoic acid (0.132 g, 88%) as a white solid.

### (3) Methyl N-[6-(benzylamino)-2-naphthoyl]phenylalaninate

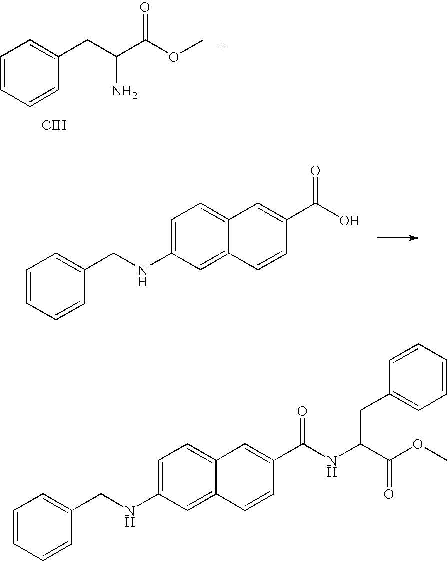

A mixture of 6-(benzylamino)-2-naphthoic acid (0.036 g, 0.13 mmol), DL-phenylalanine hydrochloride salt (0.028 g, 0.13 mmol), EDCI (0.032 g, 0.17 mmol), 1-hydrobenzotriazole (0.023 g, 0.17 mmol) and triethylamine (0.017 g, 0.16 mmol) in DMF (0.6 mL) was stirred at room temperature overnight. The mixture was partitioned between water and ethyl acetate. The separated organic phase was washed with brine, dried over sodium sulfate, filtered and concentrated under reduced pressure. The crude product was purified by preparative TLC (dichloromethane:methanol, 10:1) to give methyl N-[6-(benzylamino)-2-naphthoyl]phenylalaninate (0.038 g, 69%) as a white powder.

### (4) N-[6-(benzylamino)-2-naphthoyl]phenylalanine

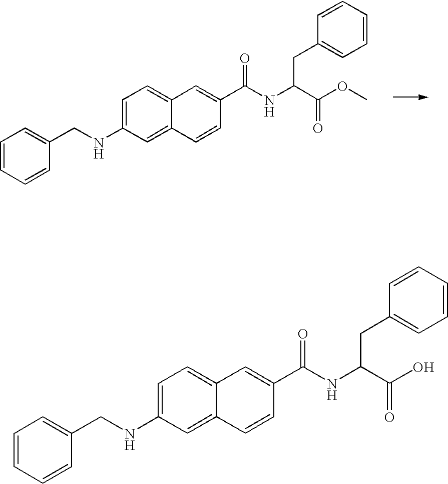

To a solution of methyl N-[6-(benzylamino)-2-naphthoyl]phenylalaninate (0.038 g, 0.09 mmol) in tetrahydrofuran (1 mL), ethanol (0.5 mL) and water (1 mL) was added lithium hydroxide monohydrate (0.011 g, 0.26 mmol), and the mixture was stirred at 60° C. for 2 hours. After cooled to room temperature, the mixture was neutralized with 1N hydrochloric acid solution (0.26 mL) and the solution was extracted with ethyl acetate. The separated organic phase was washed with water and brine, dried over sodium sulfate, filtered and concentrated under reduced pressure. The crude product was purified by recrystallization (ethyl acetate-hexane) to give N-[6-(benzylamino)-2-naphthoyl]phenylalanine (0.020 g, 53%) as a white solid.

Melting point: 172° C.

Molecular weight: 424.50

Mass spectrometry: 425

Activity grade assay 2: B

¹H-NMR (500 MHz, DMSO-d6): δ 3.09 (1H, dd, J=10.1, 13.9 Hz), 3.19 (1H, dd, 4.4, 13.9 Hz), 4.39 (2H, d, 5.7 Hz), 6.22 (1H, m), 6.71 (1H, d, 1.6 Hz), 6.82 (1H, t, 5.7 Hz), 7.10 (1H, dd, J=2.2, 8.8 Hz), 7.17 (1H, m), 7.21-7.27 (3H, m), 7.28-7.36 (4H, m), 7.41 (2H, d, J=7.3 Hz), 7.49 (1H, d, J=8.8 Hz), 7.67 (2H, d, J=9.1 Hz), 8.15 (1H, s), 8.53 (1H, d, J8.2 Hz), 12.7 (1H, Br).

1) A 2-naphthamide derivative of the formula (1), its tautomeric or stereoisomeric form, or a salt thereof: 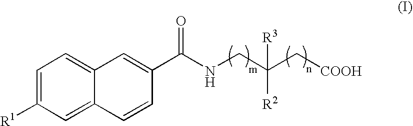
wherein
m and n independently represent an integer from 0 to 2;
—R¹ represents —O—R¹⁰—OR¹¹, —OR¹¹, —SR¹¹, —S(O)R¹¹, —S(O)₂R¹¹, —NR¹²R¹³, or —CHR¹⁴R¹⁵,
wherein
—R¹⁰— represents (C₁₋₆)alkylene;
R¹¹ represents aryl, (C₂₋₆)alkenyl optionally substituted by aryl or heteroaryl, (C₂₋₆)alkynyl optionally substituted by aryl or heteroaryl, or (C₁₋₆)alkyl optionally substituted by (C₃₋₈)-cycloalkyl, aryl or heterocycle comprising 4-9 carbons and at least one N, O, or S as a heteroatom,
wherein
said (C₃₋₈)cycloalkyl, aryl and heterocycle optionally have one or two substituents selected from the group consisting of halogen, hydroxy, nitro, (C₁₋₆)alkyl optionally substituted by mono-, di-, or tri halogen, and (C₁₋₆)alkoxy optionally substituted by (C₃₋₈)cycloalkyl, or mono-, di-, or tri halogen;
R¹² and R¹³ independently represent hydrogen, (C₂₋₆)alkenyl optionally substituted by aryl or heteroaryl, (C₂₋₆)alkynyl optionally substituted by aryl or heteroaryl, or (C₁₋₆)alkyl optionally substituted by aryl or heteroaryl,
or
R¹² and R¹³ form, together with the nitrogen atom, a 5-7 membered saturated hetero ring optionally interrupted by O or NH;
R¹⁴ and R¹⁵ independently represent hydrogen, (C₂₋₆)alkenyl optionally substituted by aryl or heteroaryl, (C₂₋₆)alkynyl optionally substituted by aryl or heteroaryl, (C₁₋₆)alkyl optionally substituted by aryl or heteroaryl, or (C₁₋₆)alkoxy optionally substituted by aryl or heteroaryl,
or
R¹⁴ and R¹⁵ form, together with the CH, a (C₃₋₈)cycloalkyl optionally interrupted by NH, or O, or a phenyl optionally substituted by hydroxy, halogen or (C₁₋₆)alkyl;
R² represents hydrogen, hydroxy, cyano, (C₁₋₆)alkoxy, (C₂₋₆)alkenyl, (C₂₋₆)alkynyl, (C₃₋₇)cycloalkyl, or (C₁₋₆)alkyl optionally having one or two substituents selected from the group consisting of hydroxy, amino, (C₁₋₆)alkylamino, aryl, and heteroaryl comprising 4-10 carbons and at least one N, O, or S as a heteroatom,
wherein
said aryl and heteroaryl optionally have one or two substituents selected from the group consisting of halogen, hydroxy, nitro, amino, N((C₁₋₆)alkyl sulfonyl)amino, morpholino, phenyl, pyridyl, (C₁₋₆)alkoxy optionally substituted by mono-, di-, or tri halogen, and (C₁₋₆)alkyl optionally substituted by mono-, di-, or tri halogen; and
R³ represents hydrogen, or (C₁₋₆)alkyl.

2) A 2-naphthamide derivative of the formula (I′), its tautomeric or stereoisomeric form, or a salt thereof: 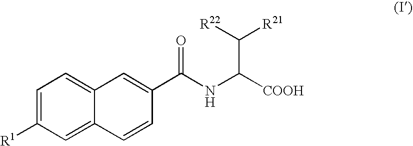
wherein
—R¹ represents —O—R¹⁰—OR¹¹, —OR¹¹, —SR¹¹, —S(O)R¹¹, —S(O)₂R¹¹, —NR¹²R¹³, or —CHR¹⁴R¹⁵,
wherein
—R¹⁰— represents (C₁₋₆)alkylene;
R¹¹ represents aryl, (C₂₋₆)alkenyl optionally substituted by aryl or heteroaryl, (C₂₋₆)alkynyl optionally substituted by aryl or heteroaryl, or (C₁₋₆)alkyl optionally substituted by (C₃₋₈)-cycloalkyl, aryl or heterocycle comprising 4-9 carbons and at least one N, O, or S as a heteroatom
wherein
said (C₃₋₈)cycloalkyl, aryl and heterocycle optionally have one or two substituents selected from the group consisting of halogen, hydroxy, nitro, (C₁₋₆)alkyl optionally substituted by mono-, di-, or tri halogen, and (C₁₋₆)alkoxy optionally substituted by (C₃₋₈)cycloalkyl, or mono-, di-, or tri halogen;
R¹² and R¹³ independently represent hydrogen, (C₂₋₆)alkenyl optionally substituted by aryl or heteroaryl, (C₂₋₆)alkynyl optionally substituted by aryl or heteroaryl, or (C₁₋₆)alkyl optionally substituted by aryl or heteroaryl,
or
R¹² and R¹³ form, together with the nitrogen atom, a 5-7 membered saturated hetero ring optionally interrupted by O or NH;
R¹⁴ and R¹⁵ independently represent hydrogen, (C₂₋₆)alkenyl optionally substituted by aryl or heteroaryl, (C₂₋₆)alkynyl optionally substituted by aryl or heteroaryl, (C₁₋₆)alkyl optionally substituted by aryl or heteroaryl, or (C₁₋₆)alkoxy optionally substituted by aryl or heteroaryl,
or
R¹⁴ and R¹⁵ form, together with the CH, a (C₃₋₈)cycloalkyl optionally interrupted by NH, or O, or a phenyl optionally substituted by hydroxy, halogen or (C₁₋₆)alkyl;
R²¹ represents hydroxy, cyano, amino, (C₁₋₆)alkylamino, thienyl, pyridyl, phenyl, naphthyl, 1H-pyrrolo[2,3-b]pyridin-3-yl, or indolyl optionally substituted by halogen or hydroxy,
wherein
said phenyl and naphthyl optionally have one or two substituents selected from the group consisting of halogen, hydroxy, nitro, ammo, N((C₁₋₆)alkyl)amino, di(C₁₋₆)alkylamino, N((C₁₋₆)alkyl sulfonyl)-amino, morpholino, phenyl, pyridyl, (C₁₋₆)alkoxy optionally substituted by mono-, di-, or tri halogen, and (C₁₋₆)alkyl optionally substituted by mono-, di-, or tri halogen; and
R²² represents hydrogen or hydroxy.

3) The 2-naphthamide derivative, its tautomeric or stereoisomeric form, or a salt thereof as claimed in claim 1 or 2,
wherein
R¹ represents phenoxy, (C₁₋₆)alkoxy optionally substituted by cyclo-propyl, cyclohexyl, pyrrolidinyl, piperidinyl, imidazolyl, pyridyl, pyrrolyl, thiazolyl optionally substituted by (C₁₋₆)alkyl, or phenyl,
wherein
said phenyl optionally has one or two substituents selected from the group consisting of fluoro, chloro, bromo, nitro, hydroxy, (C₁₋₆)alkyl optionally substituted by mono-, di, or tri halogen, and (C₁₋₆)alkoxy optionally substituted by mono-, di, or tri halogen, cyclopropyl, or cyclohexyl.

4) The 2-naphthamide derivative, its tautomeric or stereoisomeric form, or a salt thereof as claimed in claim 1 or 2,
wherein
R¹ represents phenoxy(C₁₋₆)alkyl, phenoxy(C₁₋₆)alkenyl, phenoxy(C₁₋₆)-alkynyl, or phenyl(C₁₋₆)alkoxy.

5) The 2-naphthamide derivative, its tautomeric or stereoisomeric form, or a salt thereof as claimed in claim 1,
wherein
R² represents phenyl (C₁₋₆)alkyl,
wherein
said phenyl optionally has one or two substituents selected from the group consisting of fluoro, chloro, bromo, iodo, hydroxy, nitro, amino, N(methanesulfonyl)amino, morpholino, phenyl, pyridyl, methoxy, ethoxy, and trifluoromethyl.

6) The 2-naphthamide derivative, its tautomeric or stereoisomeric form, or a salt thereof as claimed in claim 2,
wherein
R₁ represents phenoxy, (C₁₋₆)alkoxy optionally substituted by cyclo-propyl, cyclohexyl, pyrrolidinyl, piperidinyl, imidazolyl, pyridyl, pyrrolyl, phenyl, or thiazolyl optionally substituted by (C₁₋₆)alkyl,
wherein
said phenyl has optionally one or two substituents selected from the group consisting of fluoro, chloro, bromo, nitro, hydroxy, (C₁₋₆)alkyl optionally substituted by mono-, di, or tri halogen, and (C₁₋₆)alkoxy optionally substituted by mono-, di, or tri halogen, cyclopropyl, or cyclohexyl;
R²¹ represents cyano, thienyl, pyridyl, phenyl, naphthyl, 1H-pyrrolo[2,3-b]pyridin-3-yl, or indolyl optionally substituted by halogen or hydroxy,
wherein
said phenyl and naphthyl have one or two substituents selected from the group consisting of fluoro, chloro, bromo, hydroxy, nitro, amino, N((C₁₋₆)alkyl)amino, di(C₁₋₆)alkylamino, N((C₁₋₆)alkyl sulfonyl)-amino, morpholino, phenyl, pyridyl, trifluoromethyl, trifluoro-methyloxy, (C₁₋₆)alkoxy, and (C₁₋₆)alkyl; and
R²² represents hydrogen or hydroxy.

7) The 2-naphthamide derivative, its tautomeric or stereoisomeric form, or a salt thereof as claimed in claim 1 or 2,
wherein
R¹² and R¹³ independently represent hydrogen, or (C₁₋₆)alkyl optionally substituted by phenyl, naphthyl or pyridyl.

8) The 2-naphthamide derivative, its tautomeric or stereoisomeric form, or a salt thereof as claimed in claim 1, wherein said derivative is selected from the group consisting of the following compounds:
N-[6-(benzyloxy)-2-naphthoyl]phenylalanine;
N-[6-(benzyloxy)-2-naphthoyl]-4-(trifluoromethyl)phenylalanine;
N-{6-[(4-fluorobenzyl)oxy]-2-naphthoyl}phenylalanine;
N-{6-[(3-fluorobenzyl)oxy]-2-naphthoyl}phenylalanine;
N-{6-[(2-fluorobenzyl)oxy]-2-naphthoylphenylalanine;
N-[6-(3-pyridinylmethoxy)-2-naphthoyl]phenylalanine;
N-{6-[(3,4-difluorobenzyl)oxy]-2-naphthoyl}phenylalanine;
N-{6-[2-(1H-pyrrol-1-yl)ethoxy]-2-naphthoyl}phenylalanine;
N-[6-(4-pyridinyhnethoxy)-2-naphthoyl]phenylalanine;
N-[6-(benzyloxy)-2-naphthoyl]-3-(trifluoromethyl)phenylalanine;
N-[6-(benzyloxy)-2-naphthoyl]tryptophan;
N-[6-(benzyloxy)-2-naphthoyl]-O-methyltyrosine;
N-[6-(benzyloxy)-2-naphthoyl]-3-methoxytyrosine;
N-[6-(benzyloxy)-2-naphthoyl]-β-hydroxyphenylalanine;
N-[6-(2-phenylethoxy)-2-naphthoyl]phenylalanine;
N-[6-(benzyloxy)-2-naphthoyl]-4-chlorophenylalanine;
N-[6-(benzyloxy)-2-naphthoyl]-3-fluorophenylalanine;
N-{6-[(2-chlorobenzyl)oxy]-2-naphthoyl}phenylalanine;
N-{6-[(3-chlorobenzyl)oxy]-2-naphthoyl}phenylalanine;
N-{6-[(2-methoxybenzyl)oxy]-2-naphthoyl}phenylalanine;
N-{6-[(3-methoxybenzyl)oxy]-2-naphthoyl}phenylalanine;
N-{6-[(2,3-dichlorobenzyl)oxy]-2-naphthoyl}phenylalanine;
N-{6-[(3,5-dichlorobenzyl)oxy]-2-naphthoyl}phenylalanine;
N-{6-[(3,5-dimethoxybenzyl)oxy]-2-naphthoyl}phenylalanine;
N-[6-(benzyloxy)-2-naphthoyl]-3-(2-thienyl)alanine;
N-[6-(benzyloxy)-2-naphthoyl]-4-bromophenylalanine;
N-[6-(benzyloxy)-2-naphthoyl]-4-nitrophenylalanine;
N-[6-(benzyloxy)-2-naphthoyl]-3-hydroxyphenylalanine;
N-[6-(benzyloxy)-2-naphthoyl]-3-(1-naphthyl)alanine;
N-[6-(benzyloxy)-2-naphthoyl]-5-hydroxytryptophan;
N-[6-(benzyloxy)-2-naphthoyl]-2-fluorophenylalanine;
N-{6-[(2-bromobenzyl)oxy]-2-naphthoyl}phenylalanine;
N-{6-[(3-bromobenzyl)oxy]-2-naphthoyl}phenylalanine;
N-{6-[(2-methylbenzyl)oxy]-2-naphthoyl}phenylalanine;
N-{6-[(3-methylbenzyl)oxy]-2-naphthoyl}phenylalanine;
N-{6-[(3-nitrobenzyl)oxy]-2-naphthoyl}phenylalanine;
N-[6-(benzyloxy)-2-naphthoyl]-3-(2-naphthyl)alanine;
N-[6-(benzyloxy)-2-naphthoyl]-4-iodophenylalanine;
N-[6-(benzyloxy)-2-naphthoyl]-5-fluorotryptophan;
N-[6-(benzyloxy)-2-naphthoyl]-3-(1H-pyrrolo[2,3-b]pyridin-3-yl)alanine;
N-{6-[2-(4pyridinyl)ethoxy]-2-naphthoyl}phenylalanine;
N-{6-[(3-ethoxybenzyl)oxy]-2-naphthoyl}phenylalanine; and
N-[6-(2-phenylpropoxy)-2-naphthoyl]phenylalanine;

9) A medicament comprising the 2-naphthamide derivative, its tautomeric or stereoisomeric form, or a physiologically acceptable salt thereof as claimed in claim 1 as an active ingredient.

10) The medicament as claimed in claim 9, further comprising one or more pharmaceutically acceptable excipients.

11) The medicament as claimed in claim 9, wherein the 2-naphthamide derivative, its tautomeric or stereoisomeric form, or a physiologically acceptable salt thereof is an IP receptor antagonist.

12) The medicament as claimed in claim 9 for prophylaxis and/or treatment of urological disorder or disease.

13) The medicament as claimed in claim 9 for prophylaxis and/or treatment of pain.

14) The medicament as claimed in claim 9 for prophylaxis and/or treatment of hypotension.

15) The medicament as claimed in claim 9 for prophylaxis and/or treatment of hemophilia and hemorrhage.

16) The medicament as claimed in claim 9 for prophylaxis and/or treatment of inflammation.

17) Use of compounds according to claim 1 for manufacturing a medicament for the treatment and/or prophylaxis of urological disorders.

18) Use of compounds according to claim 1 for manufacturing a medicament for the treatment and/or prophylaxis of pain.

19) Use of compounds according to claim 1 for manufacturing a medicament for the treatment and/or prophylaxis of hypotension.

20) Use of compounds according to claim 1 for manufacturing a medicament for the treatment and/or prophylaxis of hemophilia and hemorrhage.

21) Use of compounds according to claim 1 for manufacturing a medicament for the treatment and/or prophylaxis of inflammation.

22) Process for controlling urological disorders in humans and animals by administration of an IP receptor-antagonisticly effective amount of at least one compound according to claim 1.

23) Process for controlling pain in humans and animals by administration of an IP receptor-antagonisticly effective amount of at least one compound according to claim 1.

24) Process for controlling hypotension in humans and animals by administration of an IP receptor-antagonisticly effective amount of at least one compound according to claim 1.

25) Process for controlling hemophilia and hemorrhage in humans and animals by administration of an IP receptor-antagonisticly effective amount of at least one compound according to claim 1.

26) Process for controlling inflammation in humans and animals by administration of an IP receptor-antagonisticly effective amount of at least one compound according to claim 1.
